(19) United States
(12) Patent Application Publication
(10) Pub. No.: US 2024/0005179 A1
(43) Pub. Date: Jan. 4, 2024

(54) COMPUTATIONAL ARCHITECTURE TO GENERATE REPRESENTATIONS OF MOLECULES HAVING TARGETED PROPERTIES

(71) Applicants: Rose Yu, Oakland, CA (US); Peter Eckmann, Oakland, CA (US); Kunyang Sun, Oakland, CA (US); Bo Zhao, Oakland, CA (US); Mudong Feng, Oakland, CA (US); Michael K. Gilson, San Diego, CA (US)

(72) Inventors: Rose Yu, Oakland, CA (US); Peter Eckmann, Oakland, CA (US); Kunyang Sun, Oakland, CA (US); Bo Zhao, Oakland, CA (US); Mudong Feng, Oakland, CA (US); Michael K. Gilson, San Diego, CA (US)

(21) Appl. No.: 18/336,696

(22) Filed: Jun. 16, 2023

Related U.S. Application Data
(60) Provisional application No. 63/357,784, filed on Jul. 1, 2022.

(51) Int. Cl. G06N 5/022; G16B 15/30; G16B 15/20; G16B 40/20

(52) U.S. Cl. CPC G06N 5/022; G16B 40/20; G16B 15/20; G16B 15/30

(57) ABSTRACT

Apparatuses, systems, and techniques are described to generate representations of molecules having one or more targeted properties. In one or more examples, an autoencoder can be trained to generate a latent space that represents a number of molecules. The latent space can be used to train a property prediction network that includes a number of property predictors. A latent space optimization process can use the property predictors to identify regions of the latent space that represent molecules having the one or more targeted properties.

### PRIORITY CLAIM

This application claims priority to U.S. Provisional Patent Application Ser. No. 63/357,784, entitled “Algorithm for de novo drug discovery (Latent Inceptionism on Molecules” filed on Jul. 1, 2022, which is incorporated by reference herein in its entirety.

### STATEMENT REGARDING FEDERALLY SPONSORED RESEARCH

This invention was made with U.S. Government support under Agreement No. GM061300, awarded by National Institute of General Medical Sciences and National Science Foundation Grant No. 2037745. The U.S. Government has certain rights in the invention.

### BACKGROUND

Drug discovery involves the identification of treatments for biological conditions that have detrimental effects on subjects in which the respective biological conditions, such as one or more diseases are present. In at least some cases, drug discovery can include identifying treatment molecules that have an affinity for a target molecule, where the target molecule has a role in causing and/or propagating disease within subjects. When a treatment molecule binds to a target molecule the function(s) of the target molecule can be disrupted resulting in a decrease or elimination of symptoms of a disease. Typically, drug discovery is costly and time consuming and can take many months, if not longer, to identify treatment molecules for a given biological condition. In addition, there can be instances where a potential treatment molecule that initially seemed to have promising results with respect to a biological condition does not provide the expected outcomes leading to the drug discovery process regressing for the biological condition.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example framework to generate representations of molecules having targeted properties, according to one or more example implementations.

FIG. 2 illustrates an example framework of a computational architecture to train an autoencoder and one or more property predictors to generate molecule representations that correspond to a target molecule, according to one or more example implementations.

FIG. 3 illustrates an example framework to identify representations of molecules located in a latent space based on targeted properties, according to one or more example implementations.

FIG. 4 is a flow diagram of an example process to generate representations of molecules having targeted properties, according to one or more example implementations.

FIG. 5 illustrates a diagrammatic representation of a machine in the form of a computer system within which a set of instructions may be executed for causing the machine to perform any one or more of the methodologies or processes discussed herein, according to one or more example implementations.

FIG. 6 is a block diagram illustrating an example architecture of software, which can be installed on any one or more of the devices described herein.

FIG. 7 illustrates a table including a comparison of QED and p-log P maximization methods. “LL” (length limit) denotes whether a model has a limited output length (about the maximum molecule size of ZINC250k), as p-log P score can increase linearly with molecule length.

FIG. 8 illustrates a table including property targeting to −2.5<log P<−2. Success (%): percent of generated molecules within the target range. Diversity: One minus the average pairwise Tanimoto similarity between Morgan fingerprints.

FIG. 9 illustrates a table including similarity-constrained p-log P maximization. For each method and minimum similarity constraint, the mean t standard deviation of improvement (among molecules that satisfy the similarity constraint) from the starting molecule is shown, as well as the percent of optimized molecules that satisfy the similarity constraint (% succ.).

FIG. 10 illustrates examples of LIMO's extremization of log P while keeping a substructure (denoted in red) constant.

FIG. 11 illustrates a table indicating generation of molecules with high computed binding affinities (shown as dissociation constants, K_{D}, in nanomoles/liter) for two protein targets, ESR1 and ACAA1.

FIG. 12 illustrates generated molecules from multi-objective (top row) and single-objective (bottom row) computed binding affinity maximization. The dissociation constant K_{D} is a measure of binding affinity, lower is better. In the bottom row, we highlight two major problematic patterns that appeared when only considering computed binding affinity, motivating multi-objective optimization.

FIG. 13 illustrates 3D visualization of ligands docked against ESR1 and ACAA1. LIMO-generated ligands (one for each protein) are shown in yellow, and raloxifene, a cancer drug that targets ESR1, is shown in pink. The protein pocket is displayed as semi-opaque, and nearby structures of the protein are shown in blue. Favorable atom-atom interactions between ligand and protein are shown with a dashed yellow line.

FIG. 14 illustrates a table showing a comparison of generated ligands for ESR1 and ACAA1 following multi-objective optimization and refinement. Arrows indicate whether a high score (″) or low score (#) is desired. High QED, Fsp3, and satisfying Lipinski's Rule of 5 suggests drug-likeness. A low number of PAINS alerts indicate specific reactivity. MCE-18 is a measure of molecular novelty based on complexity, and SA is a measure of synthesizability. KD values in nM are computed binding affinities, (AD) from AutoDock-GPU and (ABFE) from absolute binding free energy calculations.

FIG. 15 illustrates a table showing random generation of molecules trained on the MOSES dataset and calculated with the MOSES platform. % valid is the percentage of molecules that are chemically valid. U@1K is the percentage of 1,000 generated molecules that are unique. U@10K is the percentage of 10,000 generated molecules that are unique. Diversity: one minus average is the pairwise similarity between molecules. % novel is the percentage of valid generated molecules not present in training set.

FIG. 16 describes distribution of molecular properties of randomly generated molecules (Random), after performing optimization on all three properties (Opt with), and after performing optimization on the two other properties, leaving out the one on the x-axis (Opt without). For QED and SA, cutoff values are shown for the minimum and maximum (respectively) scores that we consider sufficient for further optimization. For the KD distributions, arrows mark the minimum value of each. On the x-axis, (#) indicates that a low value is desired, and (″) indicates that a high value is desired.

### DETAILED DESCRIPTION

The process to identify treatments for biological conditions typically takes many years and can cost tens of millions to hundreds of millions up to billions of dollars to identify a therapeutic that can effectively treat a disease. Identifying compounds that can bind to a target protein can be challenging because the pool of potential molecules is on the order of 10³³ or more and, typically, a small fraction from among the potential pool of molecules that bind effectively to a given target is small. Many existing treatment discovery processes include large compound screens and iteratively synthesizing and testing compounds to identify a compound that can effectively treat a biological condition.

In an attempt to reduce the amount of time and resources needed to identify molecules that can be used in candidate compounds for the treatment of a biological condition, machine learning techniques have been employed to reduce the amount of experimentation needed to analyze millions of compounds that can potentially be used to treat a given biological condition. Existing machine learning techniques for identifying candidate molecules to be used in treating a biological condition, such as generative machine learning models, reinforcement learning machine learning models, and genetic algorithms, can be computationally expensive because these models can use large amounts of processing resources and memory resources and can often be time consuming to implement. Additionally, existing machine learning techniques to identify candidate molecules to be used in treating a biological condition can produce molecules that may bind to a target protein, but that are not suitable to being used as treatments because of toxicity to humans, an inability to manufacture compounds that include the molecules, and/or because of various characteristics of the molecules that make them unsuitable for being implemented as a treatment to a biological condition, such as high molecular weights, poor solubility in carrier compounds and the like.

The systems, processes, techniques, and methods described herein are different from existing techniques in that fewer computational resources are used to identify potential target molecules for treating a biological condition. In addition, the systems, processes, techniques, and methods described herein are different from existing techniques in that a given substructure can be maintained in the candidate molecules and in that molecules with desirable properties can be more readily identified. For example, implementations described herein include an autoencoder coupled with a group of property predictors, where individual property predictors can be implemented to generate values for candidate molecules of one or more desired properties. The property predictors can be trained using output from the decoded latent space produced by the autoencoder in contrast to existing techniques that train property predictors directly from the latent space. In this way, the values for the desired properties are more favorable for treatments of a biological condition than molecules produced by existing computational techniques. In addition, the use of output from the decoded latent space produced by the autoencoder to train the property predictors can enable the identification of a fixed substructure of candidate molecules that is not performed by existing techniques and/or is less efficient in existing because the existing techniques directly train property predictors from the latent space.

Further, implementations described herein train the property predictors after the training of the autoencoder. A greater amount of data is typically used to train the generative models than the property predictors and existing techniques typically train the generative models and the property predictors at the same time. As a result, a greater amount of data than necessary is used to train the property predictors in existing techniques which leads to a greater usage of computational resources than is necessary. In contrast, by training the property predictors separately from the autoencoder, implementations described herein use less data and fewer computational resources to train the property predictors than existing techniques. In addition, the use of molecules represented by the latent space to train the property predictors rather than using a pre-existing training data set, which is typically used in existing techniques, provides a broader and more diverse training data set for the property predictors resulting in implementations described herein generating representations of molecules that have more favorable values for a set of target properties.

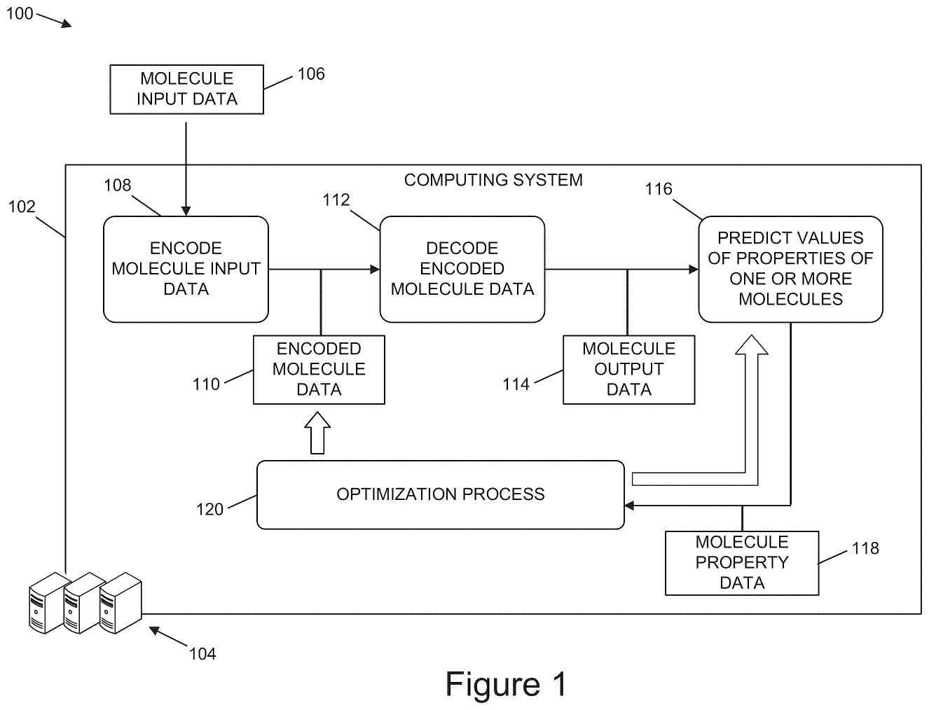

FIG. 1 illustrates an example framework 100 to generate representations of molecules having targeted properties, according to one or more example implementations. As used herein, a biological condition can refer to an abnormality of function and/or structure in an individual to such a degree as to produce or threaten to produce a detectable feature of the abnormality. A biological condition can be characterized by external and/or internal characteristics, signs, and/or symptoms that indicate a deviation from a biological norm in one or more populations. In one or more additional examples, a biological condition can include at least one of one or more diseases, one or more disorders, one or more injuries, one or more syndromes, one or more disabilities, one or more infections, one or more isolated symptoms, or other atypical variations of biological structure and/or function of individuals. Additionally, a treatment, as used herein, can refer to a substance, procedure, routine, device, and/or other intervention that can administered or performed with the intent of treating one or more effects of a biological condition in an individual. In one or more examples, a treatment may include a substance that is metabolized by the individual. The substance may include a composition of matter, such as a pharmaceutical composition. The substance may be delivered to the individual via a number of methods, such as ingestion, injection, absorption, or inhalation. A treatment may also include physical interventions, such as one or more surgeries. In at least some examples, the treatment can include a therapeutically meaningful intervention.

The framework 100 can include a computing system 102. The computing system 102 can generate representations of molecules that have one or more target properties. In one or more examples, the one or more target properties can correspond to an affinity for one or more target molecules. In one or more additional examples, the one or more target properties can correspond to similarity to molecules that are existing treatments for one or more biological conditions. Further, the one or more target properties can correspond to a likelihood of producing the one or more target molecules using one or more biomanufacturing processes. In one or more illustrative examples, the one or more target properties can correspond to threshold values for one or more physical properties of the one or more target molecules, one or more chemical properties of one or more target molecules, or a combination thereof. In at least some scenarios, the threshold values can correspond to at least one of minimum values of one or more first target properties or maximum values of one or more second target properties.

In various examples, the computing system 102 can generate representations of molecules that have previously been undiscovered in relation to treatment of one or more biological conditions. For example, the computing system 102 can be implemented as part of de novo drug discovery for one or more biological conditions. In one or more examples, the representations of molecules generated by the computing system 102 can be candidate molecules for treatments for one or more biological conditions. The candidate molecules can be synthesized and undergo a drug discovery process that determines a level of manufacturability of the candidate molecules and determines an efficacy for treatment of the one or more biological conditions. In at least some examples, the drug discovery process can include one or more in vitro processes, one or more in vivo processes, or one or more combinations thereof to determine a level of manufacturability of one or more candidate molecules. In one or more illustrative examples, clinical trials can be conducted to determine the efficacy of the candidate molecules for treatment of the one or more biological conditions.

The evaluation of candidate molecules that correspond to representations generated by the computing system 102 can include at least one of target validation, pharmacokinetic profiling, or pharmacological profiling. Assessment of the activity of candidate molecules can be based on the one or more biological conditions to be treated by the candidate molecules and/or the cells impacted by the one or more biological conditions, such as one or more organs in which symptoms of the one or more biological conditions are manifested. The evaluation of candidate molecules can include combining differentiated cells related to a biological condition with a candidate molecule or a compound that includes the candidate molecule. An investigator can determine the effect of the candidate molecule by identifying changes in the morphology, phenotype, and/or functional activity of the cells that is attributable to the target molecule in relation to other cells that are not treated with the target molecule or are treated with an inert or placebo compound, and then correlates the effect of the target molecule with the identified change(s). The diseased phenotype as well as the phenotypic changes effected by the candidate molecule on the in vitro differentiated cells can be assessed by one or more techniques. For example, the effects of a candidate molecule with respect to a biological condition can be determined by evaluating the changes to the cells treated with the candidate molecule using electron microscopy, one or more assays, one or more immunohistochemical techniques, one or more immunofluorescence techniques, analysis of the expression of nucleic acids in diseased cells, such as deoxyribonucleic acid (DNA) molecules and/or ribonucleic acid (RNA) molecules, or one or more combinations thereof. In one or more illustrative examples, the impact of a target molecule on diseases cells can be determined using one or more assays that detect the expression of one or more proteins that correspond to a given disease.

The computing system 102 can be implemented by one or more computing devices 104. The one or more computing devices 104 can include one or more server computing devices, one or more desktop computing devices, one or more laptop computing devices, one or more tablet computing devices, one or more mobile computing devices, or combinations thereof. In one or more implementations, at least a portion of the one or more computing devices 104 can be implemented in a distributed computing environment. For example, at least a portion of the one or more computing devices 104 can be implemented in a cloud computing architecture. Additionally, in at least some examples, the one or more computing devices 104 can be located in one or more locations. Further, in various examples, the one or more computing devices 104 can be at least one of controlled, maintained, or administered in relation to one or more entities, one or more organizations, one or businesses, one or more educational institutions, or one or more combinations thereof.

The computing system 102 can obtain and/or access molecule input data 106. The molecule input data 106 can include representations of one or more molecules. In at least some examples, the molecule input data 106 can include representations of one or more molecules that have at least a threshold amount of affinity for one or more target molecules. In various examples, the molecule input data 106 can include strings of characters that represent a molecule. The strings of characters can include at least one of alphanumeric characters or symbols. In one or more examples, for a given molecule, the molecule input data 106 can include text data that indicates locations of atoms for the molecule, bonds formed between atoms of the molecule, valence electrons corresponding to atoms of the molecule, cyclic arrangements of atoms of the molecule, substitutions of atoms of the molecule, locations of branches and/or side chains in relation to atoms of the molecule, or one or more combinations thereof. The molecule input data 106 can represent arrangements of atoms that correspond to valid molecules. For example, the molecule input data 106 can include alphanumeric strings that are syntactically valid by corresponding to a molecular graph and do not violate chemical rules or laws, such as the maximum number of valence electrons between atoms. In at least some examples, the character strings included in the molecule input data 106 can be formatted according to one or more formats, one or more rules, one or more syntaxes, or one or more frameworks. In one or more illustrative examples, the molecule input data 106 can include self-referencing embedded strings (SELFIES) as described in Self-Referencing Embedded Strings (SELFIES): A 100% robust molecular string representation by Krenn, Mario et al. and found at https://doi.org/10.48550/arXiv.1905.13741.

The computing system 102 can perform a number of operations to determine properties of molecules represented by the molecule input data 106. At operation 108, the computing system 102 can encode the molecule input data 106 to produce encoded molecule data 110. In one or more examples, the computing system 102 can modify the molecule input data 106 including continuous sequences of data of variable lengths having discrete values, such as a series of alphanumeric characters of symbols that represent structures of molecules, to generate the encoded molecule data 110 that includes fixed size continuous representations of the molecules. In various examples, the encoded molecule data 110 can include a latent representation of the molecule input data 106. In one or more illustrative examples, the encoded molecule data 110 can include a latent space that represents molecules as a number of nodes with individual nodes connected to at least one additional node by an edge. Individual nodes of the latent space can correspond to one or more molecules represented by a string of characters included in the molecule input data 106. In one or more additional illustrative examples, the proximity of nodes in the latent space can indicate an amount of similarity between molecules that correspond to the nodes of the latent space with respect to one or more properties of the molecules. For example, nodes of the latent space that are relatively close to each other can correspond to molecules that have a greater amount of similarity with respect to one or more properties than nodes of the latent space that are farther apart in the latent space. In at least some examples, the encoded molecule data 110 can include a number of vectors with individual vectors of the encoded molecule data 110 corresponding to one or more molecules represented by a string of characters included in the molecule input data 106.

In one or more additional illustrative examples, the encoded molecule data 110 can include a compressed version of the molecule input data 106. For example, the computing system 102 can determine a number of features for character strings included in the molecule input data 106 and determine a subset of the number of features to use to generate the encoded molecule data 110. In various examples, the features of the molecule input data 106 that are used by the computing system 102 to generate the encoded molecule data 110 can be learned through a training process performed by the computing system 102 based on a set of training data. In at least some examples, the set of training data can correspond to one or more target molecules for which the computing system 102 determines candidate treatment molecules.

In one or more examples, the computing system 102 can include one or more encoding component to perform operation 108 to generate the encoded molecule data 110 based on the molecule input data 106. In at least some examples, the computing system 102 can include one or more artificial neural networks to perform the operation 108 and generate the encoded molecule data 110 based on the molecule input data 106. In one or more illustrative examples, the computing system 102 can include an encoder of an autoencoder to perform operation 108 and generate the encoded molecule data 110 based on the molecule input data 106.

At operation 112, the computing system 102 can decode the encoded molecule data 110 to produce molecule output data 114. In one or more examples, the computing system 102 can analyze one or more nodes of a latent space that comprises the encoded molecule data 110 to determine one or more strings of characters that correspond to individual nodes of the latent space to generate the molecule output data 114. The one or more strings of characters included in the molecule output data 114 can correspond to at least one of a format, syntax, rule, or framework of the molecule input data 106. The molecule output data 114 can include text data having one or more strings of characters or variable length and having discrete values in a manner similar to or the same as that of the molecule input data 106.

In various examples, the computing system 102 can include one or more decoding components to perform operation 112 to generate the molecule output data 114 based on the encoded molecule data 110. In at least some examples, the computing system 102 can include one or more artificial neural networks to perform the operation 112 and generate the molecule output data 114 based on the encoded molecule data 110. In one or more illustrative examples, the computing system 102 can include a decoder of an autoencoder to perform operation 112 and generate the molecule output data 114 based on the encoded molecule data 110.

At operation 116, the computing system 102 can predict values of properties of one or more molecules that correspond to the molecule output data 114. In one or more examples, the computing system 102 can implement and/or access one or more property predictors to determine properties of molecules represented by the molecule output data 114. In at least some examples, the output of a decoder of the computing system 102 can be used to determine a structure of a molecule for which the computing system 102 can predict one or more properties. In one or more illustrative examples, the computing system 102 can generate visual representations of molecules that correspond to one or more strings of characters included in the molecule output data 114 and the visual representations of the molecules can be input to the one or more property predictors to determine values of properties of the molecules represented by the visual representations. In one or more additional illustrative examples, the strings of characters included in the molecule output data 114 can directly be input to the one or more property predictors to generate values of properties for molecules represented by the strings of characters included in the molecule output data 114.

In various examples, the computing system 102 can generate molecule property data 118 that includes the values of properties produced at operation 116 by the one or more property predictors. In one or more examples, the computing system 102 can include and/or have access to one or more property predictors that determine values for manufacturability of molecules represented in the molecule output data 114. In one or more additional examples, the computing system 102 can include and/or have access to one or more property predictors that determine values for affinity or binding to one or more target molecules by molecules represented in the molecule output data 114. In one or more further examples, the computing system 102 can include and/or have access to one or more property predictors that determine a measure of similarity between molecules represented by the molecule output data 114 and molecules that are treatments for one or more biological conditions.

The molecule property data 118 can be used in an optimization process 120 performed by the computing system 102. The optimization process 120 can determine molecules included in the encoded molecule data 110 that have one or more target properties. For example, the optimization process 120 can analyze the molecule property data 118 to determine molecules represented in the encoded molecule data 110 that have at least a threshold affinity for one or more target molecules. In one or more illustrative examples, the optimization process 120 can analyze molecule property data 118 for a molecule corresponding to a node in a latent space included in the encoded molecule data 110 to determine one or more additional molecules having a similar affinity or a greater affinity for one or more target molecules. In situations where the optimization process 120 is configured to identify molecules have at least a threshold affinity for one or more target molecules, the computing system 102 can determine whether the molecule property data 118 for a given molecule of the latent space included in the encoded molecule data 110 corresponds to at least the threshold affinity value for a target molecule. In situations where the molecule property data 118 includes values for the affinity of a given molecule to a target molecule that are near or above the threshold affinity, the optimization process 120 can move relatively short distances in the latent space included in the encoded molecule data 110 to identify additional nodes of the latent space that correspond to molecules that have a similar affinity value or a higher affinity value with respect to the target molecule than the given molecule. Additionally, in scenarios where the molecule property data 118 includes values for a given molecule to the target molecule that are relatively low in relation to the threshold affinity, the optimization process 120 can move relatively longer distances within the latent space included in the encoded molecule data 110 to identify further nodes of the latent space that correspond to molecules that have a higher affinity value with respect to the target molecule than the given molecule.

The optimization process 120 can also include modifying one or more atoms of a molecule represented by the molecule output data 114 to produce a modified molecule having optimal values for one or more properties included in the molecule property data 118 for the molecule. For example, the optimization process 120 can include modifying one or more carbon atoms of a molecule represented by the molecule output data 114 to be one or more heteroatoms, such as oxygen (O), nitrogen (N), or sulfur (S) in an effort to optimize values for one or more properties of the modified version of the molecule. Additionally, the optimization process 120 can including modifying one or more features of cyclical structures of a molecule represented by the molecule output data 114 to optimize values for one or more properties of the modified version of the molecule, such as at least one of modifying a number of atoms or modifying bonds between atoms included in the cyclical structure. Further, the optimization process 120 can include at least one of adding or modifying substituents to one or more atoms of a molecule represented by the molecule output data 114 to optimize values for one or more properties of the modified version of the molecule. In at least some examples, the optimization process 120 can include designating a substructure that includes a number of atoms of a molecule represented by the molecule output data 114 and modifying other atoms of the molecule outside of the substructure to optimize values for one or more properties of the modified version of the molecule.

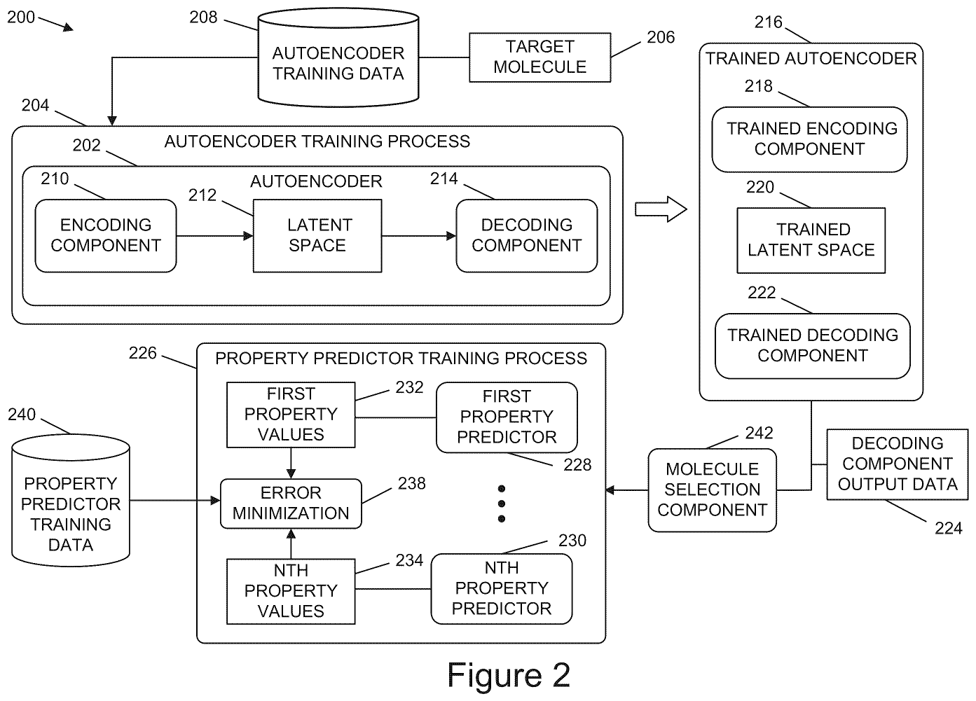

FIG. 2 illustrates an example framework 200 of a computational architecture to train an autoencoder 202 and one or more property predictors to generate molecule representations that correspond to one or more target molecules, according to one or more example implementations. In various examples, the autoencoder 202 can include a variational autoencoder. The framework 200 can include an autoencoder training process 204. The autoencoder training process 204 can train the autoencoder 202 to generate representations of molecules that correspond to a target molecule 206. In one or more examples, the target molecule 206 can include a protein. In one or more additional examples, the target molecule 206 can include an enzyme. In at least some examples, the target molecule 206 can be related to a biological condition that is present in a number of subjects. In one or more illustrative examples, the target molecule 206 can be expressed in individuals in which one or more diseases is present.

In one or more examples, the autoencoder training process 204 can be performed to train the autoencoder 202 to generate representations of molecules that have at least a threshold amount of affinity for the target molecule 206. For example, the autoencoder training process 204 can be performed to train the autoencoder 202 to generate representations of molecules that have at least a threshold probability of binding to one or more locations of the target molecule 206. In at least some scenarios, the binding of molecules to the target molecule 206 can disrupt the function of the target molecule 206. In one or more additional examples, the binding of molecules to the target molecule 206 can enhance the function of the target molecule 206. In various examples, the autoencoder training process 204 can be used to train the autoencoder 202 to generate representations of molecules that have not been previously synthesized or produced. In this way, the autoencoder training process 204 can be performed to train the autoencoder 202 to generate representations of molecules that can be used as part of a de novo drug discovery process to treat one or more biological conditions in which the target molecule 206 is present.

In one or more illustrative examples, the autoencoder training process 204 can be performed to train the autoencoder 202 to generate representations of organic molecules having a molecular weight of no greater than 1000 Daltons, no greater than 900 Daltons, no greater than 800 Daltons, no greater than 700 Daltons, no greater than 600 Daltons, no greater than 500 Daltons, or no greater than 400 Daltons. In one or more additional illustrative examples, the autoencoder training process 204 can be performed to train the autoencoder 202 to generate representations of organic molecules having a molecular weight of at least 50 Daltons, at least 100 Daltons, at least 150 Daltons, at least 200 Daltons, at least 250 Daltons, at least 300 Daltons, or at least 350 Daltons. In one or more further illustrative examples, the autoencoder training process 204 can be performed to train the autoencoder 202 to generate representations of organic molecules having a molecular weight from about 50 Daltons to about 1000 Daltons, from about 100 Daltons to 900 Daltons, from about 200 Daltons to about 800 Daltons, from about 300 Daltons to about 700 Daltons, from about 400 Daltons to about 800 Daltons, from about 500 Daltons to about 1000 Daltons, or from abut 50 Daltons to about 500 Daltons. In various examples, the autoencoder training process 204 can be performed to train the autoencoder 202 to generate representations of relatively larger molecules, such as representations of proteins that correspond to the target molecule 206.

The autoencoder training process 204 can use autoencoder training data 208 to train the autoencoder 202. The autoencoder training data 208 can include representations of a number of molecules that correspond to one or more criteria. For example, the autoencoder training data 208 can include representations of molecules that have a range of molecular weights, a range for the number of atoms included in the molecules, numbers of bonds included in the molecules, types of bonds between atoms, characterization of atoms included in the molecules (e.g., carbon, nitrogen, sulfur, oxygen, fluorine, bromine, hydrogen, etc.), cyclical structure of atoms included in the molecules, one or more physical properties of the molecules, binding affinity to target molecules, one or more combinations thereof, and the like. In one or more examples, the autoencoder training data 208 can include representations of molecules that include at least one of strings that comprise characters or symbols, molecule graphs, three-dimensional molecular representations, or binary molecule fingerprints. In one or more illustrative examples, the autoencoder training data 208 can include representations of thousands of molecules, tens of thousands of molecules, hundreds of thousands of molecules, or more than 1 million molecules. In various examples, the autoencoder training data 208 can undergo preprocessing before being sent to or accessed as part of the autoencoder training process 204. To illustrate, in scenarios where the autoencoder training data 208 includes molecular graphs and/or three-dimensional representations of molecules, the autoencoder training data 208 can be processed to generate strings of characters and/or symbols that are provided as part of the autoencoder training process 204. In at least some examples, the autoencoder training data 208 can be preprocessed such that input to the autoencoder 202 includes one-hot encoded representations of the input molecules.

The autoencoder 202 can include an encoding component 210. The encoding component 210 can include one or more artificial neural networks. In one or more examples, the encoding component 210 can include one or more computational layers with individual computational layers including a number of computational nodes that execute one or more functions and one or more weights that correspond to the individual computational nodes. At least a portion of the computational nodes of the encoding component 210 can be fully-connected layers. In one or more illustrative examples, the encoding component 210 can include one or more embedding layers that feed into a number of linear computational layers. In various examples, the encoding component 210 can implement an activation function. For example, the encoding component 210 can implement a rectified linear unit (ReLU) activation function.

The encoding component 210 can generate a latent space 212. The latent space 212 can include compressed version of the autoencoder training data 208. For example, the autoencoder training data 208 can include representations of molecules having a number of features and the autoencoder training process 204 can reduce the number of features used to represent the molecules included in the autoencoder training data 208. The latent space 212 can include the compressed representation of the features of the autoencoder training data 208. In one or more examples, the latent space can include a vector representation of features of molecules included in the autoencoder training data 208. In various examples, the autoencoder training data 208 can include representations of molecules that include strings of variable lengths and having discrete values and the latent space 212 can include a fixed size, continuous representation of the molecules included in the autoencoder training data 208. In one or more illustrative examples, the latent space 212 can include a number of nodes with individual nodes corresponding to one or more molecules included in the autoencoder training data 208. In these situations, individual nodes of the latent space 212 can be connected by edges that indicate an amount of similarity between the individual nodes. In at least some examples, the proximity of nodes in the latent space 212 can indicate an amount of similarity between one or more features of the molecules that correspond to the nodes.

The autoencoder 202 can also include a decoding component 214. The decoding component 214 can include one or more artificial neural networks. In one or more examples, the decoding component 214 can include one or more computational layers with individual computational layers including a number of computational nodes that execute one or more functions and one or more weights that correspond to the individual computational nodes. At least a portion of the computational nodes of the decoding component 214 can be fully-connected layers. In one or more illustrative examples, the decoding component 214 can include one or more embedding layers that feed into a number of linear computational layers. In various examples, the decoding component 214 can implement a Softmax function in an output layer. In at least some examples, the decoding component 214 can implement a rectified linear unit (ReLU) activation function.

In one or more examples, the autoencoder training process 204 can include performing one or more batch normalization operations with respect to at least one of the encoding component 210 or the decoding component 214. In addition, the reconstruction error of the autoencoder 202 can be minimized using one or more negative log-likelihood techniques. Further, autoencoder training process 204 can include training the autoencoder 202 over a number of epochs of the autoencoder training data 208. For example, the autoencoder training process 204 can include training the autoencoder 202 over at least 2 epochs of the autoencoder training data 208, at least 4 epochs of the autoencoder training data 208, at least 6 epochs of the autoencoder training data 208, at least 8 epochs of the autoencoder training data 208, at least 10 epochs of the autoencoder training data 208, at least 12 epochs of the autoencoder training data 208, at least 14 epochs of the autoencoder training data 208, at least 16 epochs of the autoencoder training data 208, at least 18 epochs of the autoencoder training data 208, at least 20 epochs of the autoencoder training data 208, or at least 25 epochs of the autoencoder training data 208.

After performing the autoencoder training process 204, a trained autoencoder 216 can be produced. The trained autoencoder 216 can include a trained encoding component 218, a trained latent space 220, and a trained decoding component 222. In at least some examples, the trained autoencoder 216 can include a variational autoencoder. The trained decoding component 222 can access portions of the trained latent space 220 to generate decoding component output data 224. The decoding component output data 224 can include text data comprising strings of at least one of characters or symbols that represent molecules characterized in the trained latent space 220.

The decoding component output data 224 can be used in a property predictor training process 226. For example, the property predictor training process 226 can take place after the autoencoder training process 204 is complete. To illustrate, during the training of the property predictor training process 226, the weights of the trained autoencoder 216 can be unmodified or frozen. In one or more examples, the property predictor training process 226 can be implemented to train a number of property predictors, such as a first property predictor 228 up to an Nth property predictor 230. The first property predictor 228 can generate first property values 232 that include values for a first property of molecules represented by the decoding component output data 224 and the Nth property predictor 230 can generate Nth property values 234 that include values for an Nth property of molecules represented by the decoding component output data 224.

In one or more examples, the property predictors that are trained according to the property predictor training process 226 can include one or more property predictors that generate values indicating an amount of similarity of molecules represented by the decoding component output data 224 with respect to molecules that are or have been used as treatments for one or more biological conditions. The property predictors trained according to the property predictor training process 226 can also include one or more property predictors that generate values indicating a level of manufacturability of the molecules represented by the decoding component output data 224, such as a probability that the molecules would remain intact under conditions of one or more biomanufacturing processes. Additionally, the property predictors trained according to the property predictor training process 226 can include one or more property predictors that generate values indicating an affinity for the target molecule 206. For example, at least one property predictor of the number of property predictors trained according to the property predictor training process 226 can generate values of binding affinity of molecules represented by the decoding component output data 224 with respect to one or more locations of the target molecule 206. Further, one or more property predictors trained according to the property predictor training process 226 can generate values of molecules that correspond to toxicity in humans or other subjects. In various examples, the property predictor training process 226 can be performed for different target molecules. To illustrate, a first implementation of the property predictor training process 226 can be performed with respect to the target molecule 206 and a second implementation of the property predictor training process 226 can be performed with respect to an additional target molecule.

In one or more illustrative examples, one or more property predictors trained using the property predictor training process 226 can generate values for dissociation constants to indicate an affinity for binding to the target molecule 206. For example, one or more property predictors included in the property predictor training process 226 can be implemented according to techniques described in at least one of Santos-Martins, Diogo & Solis-Vasquez, Leonardo & Tillack, Andreas & Sanner, Michel & Koch, Andreas & Forli, Stefano. (2021). Accelerating AutoDock4 with GPUs and Gradient-Based Local Search. Journal of Chemical Theory and Computation. 10.1021/acs.jctc.0c01006 or Cournia Z, Allen B K, Beuming T, Pearlman D A, Radak B K, Sherman W. Rigorous Free Energy Simulations in Virtual Screening. J Chem Inf Model. 2020 Sep. 28; 60(9):4153-4169. doi: 10.1021/acs.jcim.0c00116. Epub 2020 Jun. 16. PMID. 32536386. Further, the one or more property predictors included in the property predictor training process 226 can be implemented to generate values related to quantitative estimates of drug-likeness scores according to Bickerton G R, Paolini G V, Besnard J, Muresan S, Hopkins A L Quantifying the chemical beauty of drugs. Nat Chem. 2012 Jan. 24; 4(2):90-8. doi: 10.1038/nchem 1243. PMID: 22260643; PMCID: PMC3524573 and drug-likeness scores according to Lipinski C A, Lombardo F, Dominy B W, Feeney P J. Experimental and computational approaches to estimate solubility and permeability in drug discovery and development settings Adv Drug Deliv Rev. 2001 Mar. 1; 46(1-3).3-26. doi. 10.1016/s0169-409x(00)00129-0. PMID: 11259530. The one or more property predictors included in the property predictor training process 226 can calculate synthetic accessibility scores according to Ertl P, Schuffenhauer A. Estimation of synthetic accessibility score of drug-like molecules based on molecular complexity and fragment contributions. J Cheminform. 2009 Jun. 101(1):8. doi. 10.1186/1758-2946-1-8 PMID. 20298526; PMCID: PMC3225829 and measures of molecular complexity determined according to Ivanenkov Y A, Zagribelnyy B A, Aladinskiy V A Are We Opening the Door to a New Era of Medicinal Chemistry or Being Collapsed to a Chemical Singularity? J Med Chem. 2019 Nov. 27; 62(22):10026-10043. doi: 10.1021/acs.jmedchem 9b00004. Epub 2019 Jun. 26 PMID: 31188596. In still further examples, the one or more property predictors included in the property predictor training process 226 can include determining measures of non-specific activity against a number of biological targets according to Baell J B, Holloway G A. New substructure filters for removal of pan assay interference compounds (PAINS) from screening libraries and for their exclusion in bioassays. J Med Chem. 2010 Apr. 8; 53(7):2719-40. doi. 10.1021/jm901137j. PMID: 20131845 and determining favorable drug properties based on amounts of sp³ hybridized carbons according to Wei W, Cherukupalli S, Jing L, Liu X, Zhan P. Fsp: A new parameter for drug-likeness. Drug Discov Today. 2020 October; 25(10):1839-1845. doi: 10.1016/j.drudis.2020.07.017. Epub 2020 Jul. 24. PMID 32812310. In still additional examples, one or more property predictors included in the property predictor training process 226 can determine values for log P according to Vraka C, Nics L, Wagner K I-H, Hacker M, Wadsak W. Mitterhauser M. Log P, a yesterday's value? Nucl Med Biol. 2017 July; 50:1-10. doi: 10.1016/j.nucmedbio.2017.03.003. Epub 2017 Mar. 21. PMID: 25364662 and/or Jin, W., Barzilay, R. &amp; Jaakkola, T. (2018). Junction Tree Variational Autoencoder for Molecular Graph Generation. <i>Proceedings of the 35th International Conference on Machine Learning, in Proceedings of Machine Learning Research 80:2323-2332 Available from https://proceedings.mlr.press/v80/jin18a.html.

The property predictor training process 226 can include an error minimization process 238 that is performed by analyzing property values generated by the first property predictor 228 up to the Nth property predictor 230 in relation to property predictor training data 240. In one or more examples, the property predictor training data 240 can include a source for a ground-truth property estimation function. In various examples, the error minimization process 238 can include minimizing a mean square error of predicted property values generated by the property predictors included in the property predictor training process 226. In one or more illustrative examples, the error minimization process 238 can be performed in relation to the following loss equation:

[symbol]

₀(θ)=∥g_{θ}(f_{dec}(z)−π(f_{dec}(z))∥²,

where z corresponds to the latent space, g_{θ} corresponds to the property predictors included in the property predictor training process 226, f_{dec} corresponds to the trained decoding component 222, θ corresponds to parameters of the property predictors g_{θ}, and π corresponds to a ground-truth property estimation function.

In one or more examples, the property predictor training process 226 can implement a molecule selection component 242 that accesses molecule representations included in the trained latent space 220 and causes one or more property predictors to generate values of properties for the molecule representations. In at least some examples, the molecule selection component 242 can implement one or more random or pseudo-random processes for selecting molecule representations included in the trained latent space 220 for the property predictor training process 226. In various examples, the property predictors that are included in the property predictor training process 226 can be individually trained. The amount of data used to train the individual property predictors included in the property predictor training process 226 can be less than the amount of data used to produce the trained autoencoder 216. For example, the molecule selection component 242 can obtain decoding component output data 224 for tens of thousands up to hundreds of thousands of molecules represented by the trained latent space 220 to perform the property predictor training process 226 for at least a portion of the property predictors included in the property predictor training process 226. In still other examples, the molecule selection component 242 can obtain decoding component output data for thousands up to tens of thousands of molecules represented by the trained latent space 220 to perform the property predictor training process 226 for additional property predictors included in the property predictor training process 226, such as one or more property predictors used to generate values for binding affinity with respect to the target molecule 206. After the property predictor training process 226 is complete, the property predictors including the first property predictor 228 up to the Nth property predictor 230 can be used to identify molecules included in the trained latent space 220 having one or more targeted properties.

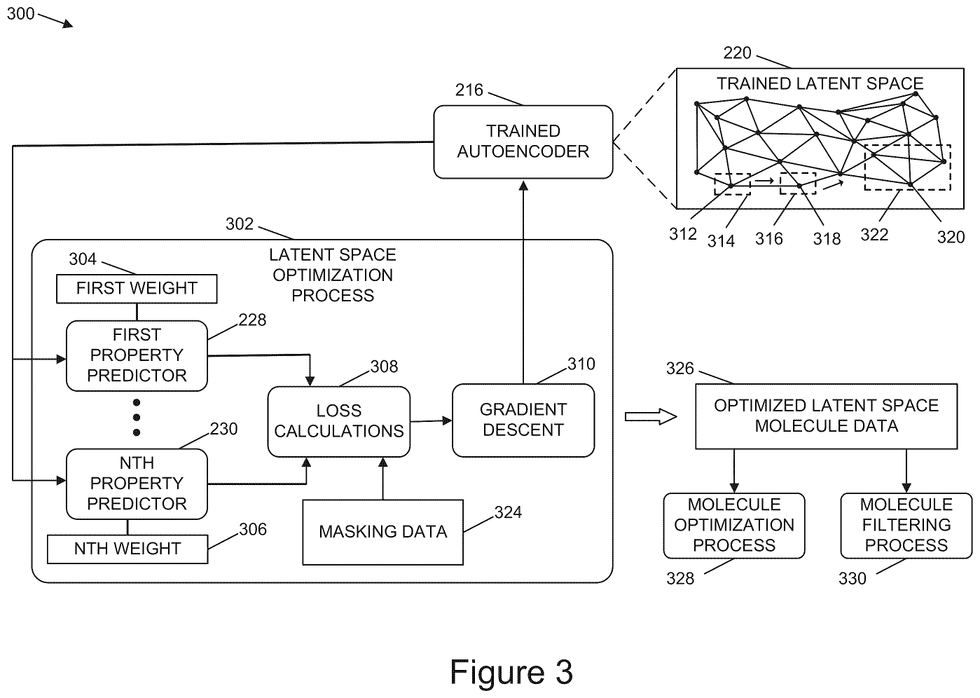

FIG. 3 illustrates an example framework 300 to identify representations of molecules located in a latent space based on targeted properties, according to one or more example implementations. The framework 300 can include the trained autoencoder 216 described with respect to FIG. 2 and the trained latent space 220 that is part of the trained autoencoder 216. The framework 300 can also include a latent space optimization process 302 that identifies molecule representations included in the trained latent space 220 that have targeted properties. The targeted properties can correspond to values of properties that satisfy one or more thresholds. For example, the targeted properties can include a minimum binding affinity with one or more target molecules. In at least some cases, the minimum binding affinity can correspond to a maximum dissociation constant for a target molecule when bound with a molecule included in the trained latent space 220. Additionally, the targeted properties can include a minimum drug-likeness score, such as a minimum QED score and/or a number of conditions satisfied under Lipinski's rule of 5. Further, the targeted properties can include a minimum manufacturability score. In various examples, the targeted properties can include threshold values in relation to the example property predictors discussed in relation to the property predictor training process 226 of FIG. 2.

In one or more examples, the latent space optimization process 302 can include determining weights for a number of property predictors with respect to identifying representations of molecules included in the trained latent space 220 that correspond to one or more targeted properties. For example, the latent space optimization process 302 can include determining a first weight 304 of the first property predictor 228 up to an Nth weight 306 of the Nth property predictor 230. In various examples, representations of molecules included in the trained latent space 220 can be accessed and/or provided to one or more of the property predictors and one or more loss calculations 308 can be performed based on the values for the respective properties generated by the property predictors for the molecules. In one or more illustrative examples, one or more gradient descent techniques 310 can be implemented to identify molecules included in the trained latent space 220 that minimize the loss calculations 308. In various examples, the loss calculations 308 used to identify molecules included in the trained latent space 220 that have one or more targeted properties can be determined according to the following equation:

[symbol]

₁(z)=−Σᵢ₌₁^{k}wᵢ·gⁱ(f_{dec}(z)),

where gⁱ corresponds to a property predictor, z corresponds to the trained latent space 220, wᵢ corresponds to a weight for a respective property predictor, and f_{dec} corresponds to the trained decoder of the trained autoencoder 216.

In one or more additional illustrative examples, implementing the gradient descent techniques 310 can include moving from one region of the trained latent space 220 to another region of the trained latent space 220 to identify representations of molecules having one or more targeted properties. For example, a molecule corresponding to a first node 312 located in a first region 314 of the trained latent space 220 can be obtained and/or accessed as part of the latent space optimization process 302. The representation of the first molecule corresponding to the first node 312 can be processed by a decoding component of the trained autoencoder 216 and the decoding component output data can be provided to one or more property predictor. The values calculated by the one or more property predictors for the first molecule can be used to generate loss calculation 308 and the gradient descent techniques 310 can be implemented to determine that a move from the first node 312 to a second node 316 located in a second region 318 of the trained latent space 220, where the second node 316 corresponds to a second molecule that has a possibility of having values of one or more properties that are closer to the one or more targeted properties than the values of the properties for the first molecule.

Continuing with the above example, a representation of the second molecule corresponding to the second node 316 can be processed by the decoding component of the trained autoencoder 216 and the decoding component output data can be provided to the one or more property predictor. The values calculated by the one or more property predictors for the second molecule can be used to generate loss calculation 308 and the gradient descent techniques 310 can be implemented to determine that a move from the second node 316 to an additional node of the trained latent space 220. As the latent space optimization process continues 302, the gradient descent techniques 310 can identify a third node 320 located in a third region 322 of the trained latent space 220, where the third node 320 corresponds to a third molecule that has values of one or more properties that correspond to the one or more targeted properties than the values of the properties for the other molecules identified and evaluated as part of the latent space optimization process 302. In the illustrative example of FIG. 3, the third region 322 can include multiple nodes that correspond to molecules having the one or more targeted properties.

In one or more examples, the latent space optimization process 302 can be performed such that a substructure is present in the molecules identified using the latent space optimization process 302. In these scenarios, the latent space optimization process 302 can be performed using masking data 324. The masking data 324 can indicate atoms of a substructure that are to remain constant during the identification of molecules by the latent space optimization process 302. In this way, the molecules in the trained latent space 220 identified using the latent space optimization process 302 that have the targeted properties include the substructure specified by the masking data 324. In one or more illustrative examples, the masking data 324 can be obtained based on user input indicating the atoms of the substructure that are to be present in the molecules identified by the latent space optimization process 302. In various examples, the loss calculations 308 can be implemented in relation to the masking data 324 according to the following equations:

[symbol]

₂(z)=λΣᵢ₌₁ⁿΣⱼ₌₁^{d}(M_{i,j}·(f_{dec}(z)_{i,j}−(\hat{x}ₛₜₐᵣₜ)_{i,j}))²,

where M_{i,j} corresponds to a symbol j at position i in the string of characters representing a molecule that is input to the one or more property predictors with M_{i,j}=1 when the desired substructure is present and M_{i,j}=0 when the symbol cannot be changed. xₛₜₐᵣₜ is a molecule having the desired substructure included in the masking data and \hat{x}ₛₜₐᵣₜ=f_{dec}(f_{enc}(xₛₜₐᵣₜ)) where f_{dec} corresponds to the decoding component of the trained autoencoder and f_{enc}(corresponds to the encoding component of the trained autoencoder. Additionally, z corresponds to the trained latent space and λ is a weighting term.

The latent space optimization process 302 can generate optimized latent space molecule data 326 that corresponds to one or more regions of the trained latent space 220 that includes representations of molecules that correspond to the one or more target properties. In at least some examples, the optimized latent space molecule data 326 can be further processed according to a molecule optimization process 328 and/or a molecule filtering process 330. The molecule optimization process 328 can include modifying one or more atoms, one or more substituents, or one or more cyclical structures of at least a portion of the molecules represented by the optimized latent space molecule data 326 to produce a set of modified molecule representations that correspond to a modified set of molecules. The modified molecule representations can then be analyzed using one or more property predictors to determine whether or not the properties of a modified molecule improve in relation to an initial molecule included in the optimized latent space molecule data 326. In one or more illustrative examples, the molecule optimization process 328 can include systematically modifying one or more carbon atoms of one or more molecules represented by the optimized latent space molecule data 326 to be one or more heteroatoms, such as oxygen, nitrogen, or sulfur, and with each modification to the one or more carbon atoms analyzing the impact on the values of one or more properties using a modified molecule representation in conjunction with one or more property predictors. In this way, in at least some instances, the molecule optimization process 328 can determine one or more modified molecules that have improved properties with respect to the properties of molecules represented by the optimized latent space molecule data 326.

The molecule filtering process 330 can include analyzing molecules represented by the optimized latent space molecule data 326 according to one or more criteria to determine a subset of molecules represented by the optimized latent space molecule data 326 that satisfy the one or more criteria. In various examples, cutoff values for one or more properties can be determined based on values of properties of molecules that are included in existing treatments for one or more biological conditions and/or for molecules that bind to one or more target proteins. For example, the molecule filtering process 330 can include determining cutoff values for molecules to include in a subset of the molecules represented by the optimized latent space molecule data 326 based on values for quantitative estimates of drug-likeness (QED) that are determined for molecules that are included in existing treatments for one or more biological conditions and/or for molecules that bind to one or more target proteins. Additionally, the molecule filtering process 330 can include determining cutoff values for molecules to include in a subset of the molecules represented by the optimized latent space molecule data 326 based on values of synthetic accessibility scores (SA) for molecules that are included in existing treatments for one or more biological conditions and/or for molecules that bind to one or more target proteins. Further, the molecule filtering process 330 can include determining cutoff values for molecules to include in a subset of the molecules represented by the optimized latent space molecule data 326 based on the size of cyclic rings included in the molecules represented by the optimized latent space molecule data 326. In one or more illustrative examples, cutoff values for the size of rings of the molecules includes in the optimized latent space molecule data 326 can include molecules with rings having no greater than 4 atoms, molecules with rings having no greater than 5 atoms, molecules with rings having no greater than 6 atoms, molecules with rings having no greater than 7 atoms, molecules with rings having no greater than 8 atoms, molecules with rings having no greater than 9 atoms, or molecules with rings having no greater than 10 atoms. In situations where molecules represented by the optimized latent space molecule data 326 satisfy the cutoff values, the molecules can be added to a subset of molecules that are candidates for being synthesized. In at least some examples, one or more molecules included in the subset of molecules can be synthesized and subjected to in vitro and/or in vivo testing to determine efficacy of the one or more molecules for the treatment of one or more biological conditions.

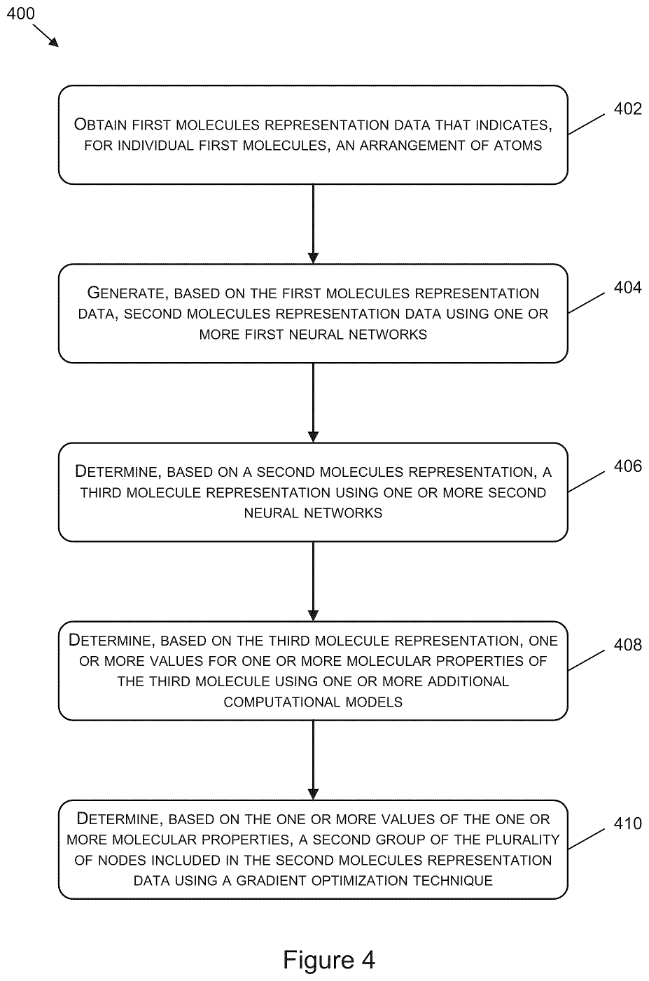

FIG. 4 illustrates example methods for generating representations of molecules having targeted properties. The example processes are illustrated as collections of blocks in logical flow graphs, which represent sequences of operations that can be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks can be combined in any order and/or in parallel to implement the process.

The process 400 can include, at 402, obtaining first molecules representation data that indicates, for individual first molecules, an arrangement of atoms. In one or more examples, the first arrangement of atoms can be represented by a first string of characters.

At 404, the process 400 can include generating, based on the first molecules representation data, second molecules representation data using one or more first neural networks. The second molecules representation data can include a plurality of nodes with a first group of the plurality of nodes individually corresponding to a vector representing a respective arrangement of atoms of an individual first molecule. In one or more examples, at least one of the first molecules or the second molecules have molecular weights no greater than 800 Daltons

In addition, the process 400 can include, at 406, determining, based on a second molecules representation, a third molecule representation using one or more second neural networks. The third molecule representation indicating an additional arrangement of atoms of a third molecule. In one or more examples, the additional arrangement of atoms can be represented using an additional string of characters. In one or more examples, the third molecule can comprise at least one heteroatom and the third molecule can be optionally polycyclic. The at least one heteroatom can be selected from nitrogen, oxygen, and sulfur. Further, the third molecule can be optionally substituted by one or more substituents.

In one or more examples, the one or more first neural networks can correspond to an encoder of a variational autoencoder and the one or more second neural networks correspond to a decoder of the variational autoencoder. In one or more examples, a training process for the variational autoencoder can be performed to minimize a reconstruction loss of the variational autoencoder. In at least some examples, a loss function of the variational autoencoder is minimized using negative log likelihood.

The process 400 can also include, at 408, determining, based on the third molecule representation, one or more values for one or more molecular properties of the third molecule using one or more additional computational models. At least one molecular property of the one or more molecular properties can indicate a binding affinity to a target molecule. In various examples, the one or more additional computational models can include a first computational model to generate one or more values of log P for the third molecule and a second computational model to generate one or more values of a dissociation constant that corresponds to the target molecule with respect to the third molecule. In addition, the one or more additional computational models can include a third computational model to generate one or more values indicating an amount of sp3 hybridized carbons of the third molecule and a fourth computational model to generate one or more values indicating one or more measures of similarity for the third molecule with respect to existing treatments for one or more biological conditions. Further, the one or more additional computational models can include a fifth computational model to generate one or more values indicating non-specific activity with respect to a plurality of biological target molecules and a sixth computational model to generate one or more values indicating a measure of molecular novelty for the third molecule.

In one or more illustrative examples, an additional computational model of the one or more additional computational models can be implemented to determine, based on one or more additional molecule representations of the plurality of additional molecule representations, first values for a first metric for one or more additional molecules that correspond to the one or more additional molecule representations. The first values of the first metric can indicate measures of similarity between the one or more additional molecules and one or more further molecules that are provided as treatments for one or more biological conditions. In one or more additional illustrative examples, an additional computational model of the one or more computational models can be used to determine, based on the one or more additional molecule representations of the plurality of additional molecule representations, second values for a second metric for the one or more additional molecules. The second values for the second metric can indicate a likelihood of synthesizing the one or more additional molecules under one or more sets of conditions. Additionally, a subset of the one or more additional molecules can be determined having at least a threshold value for the first metric and at least an additional threshold value for the second metric, where the subset of the one or more additional molecules can comprise target molecules for treating a biological condition.

The one or more additional computational models can be trained to predict binding affinity to one or more regions of the target molecule. In one or more examples, the target molecule can include a protein. For example, the protein can include an enzyme. Additionally, the one or more regions can include an active site of the target molecule. In one or more further examples, the one or more regions can include an allosteric site of the target molecule. In still other examples, the one or more regions can include an agonistic site of the target molecule. In one or more examples, the one or more regions can include an antagonistic site of the target molecule.

In one or more examples, the training process for the variational autoencoder can include obtaining a training data set indicating training molecules representation data that indicates. For individual training molecules, an arrangement of atoms can be represented using a string of characters. Additionally, the training process can include generating first training molecule representations using the encoder where individual first training molecule representations corresponding to a vector indicating a compressed version of the one or more first training molecule representations. Further, the training process for the variational autoencoder can include generating, based on the first training molecule representations, one or more second training molecule representations using the decoder. The individual second training molecule representations can indicate a modified arrangement of atoms using a modified character string. In various examples, the training process for the variational autoencoder can include analyzing the one or more second training molecule representations with respect to the training data set to determine a reconstruction loss for the variational autoencoder. In at least some examples, the training process for the variational autoencoder can include modifying, based on the reconstruction loss, one or more computational layers of at least one of the encoder or the decoder to minimize a loss function of the variational autoencoder.

An additional training process of the additional computational models used to predict the molecular properties of molecule representations can be performed that includes generating a plurality of further molecule representations using the decoder and based on a plurality of vectors extracted from the plurality of nodes. The further molecule representations can be used in the additional training process by predicting values of the one or more molecular properties of molecules that correspond to the plurality of modified molecule representations. In one or more examples, the one or more additional computational models are trained after the variational autoencoder is trained and computational layers of the variational autoencoder remain unchanged during the additional training process of the one or more additional computational models.

Further, at 410, the process 400 can include determining, based on the one or more values of the one or more properties, a second group of plurality of nodes included in the second molecules representation data using a gradient optimization technique. The second group of the plurality of nodes individually can correspond to a vector indicating an additional arrangement of atoms of an additional molecule having at least a threshold value of the at least one molecular property.

In one or more examples, a molecule optimization process can be performed to identify the portions of the second molecules representation data correspond to molecules having one or more targeted properties. In various examples, the molecule optimization process can include obtaining masking data indicating one or more positions of the one or more third molecules that correspond to a substructure that is to remain fixed during a molecule optimization process. In addition, the molecule optimization process can include generating a plurality of modified molecule representations with individual modified molecule representations indicating a modified molecule that includes the substructure and includes one or more atoms modified from a respective third molecule of the one or more third molecules. In at least some examples, the atoms being modified can be outside of the substructure. Further, the molecule optimization process can include determining, using the one or more additional computational models, that an individual modified molecule that corresponds to a modified molecule representation of the plurality of modified molecule representations that has an additional value for a molecular property of the one or more molecular properties that is greater than the value for the molecular property with respect to at least a portion of the one or more third molecules that include the substructure. In this way, the molecule optimization process can identify molecule representations that correspond to molecules having values that are closer to or satisfy values for one or more target properties. For example, the molecule optimization process with respect to the third molecule representation can includes modifying, one or more atoms of the third molecule representation to generate a respective modified molecule representation that corresponds to a respective modified molecule having an arrangement of atoms different from the third arrangement of atoms and determining, using the one or more additional computational models, a modified value for the molecular property of the modified molecule. the molecule optimization process for the third molecule representation can then include determining that the modified value for the molecular property of the modified molecule is greater than a value for the molecular property of the one or more molecular properties of the third molecule. In one or more illustrative examples, modifying the one or more atoms includes changing a carbon atom to at least one of a nitrogen atom, an oxygen atom, a chlorine atom, or a fluorine atom.

In one or more illustrative examples, the plurality of nodes can be included in a latent space that corresponds to the second molecules representation data. Additionally, a subset of the plurality of additional molecule representations can correspond to a subset of the plurality of nodes located in the latent space. The subset of the plurality of additional molecule representations correspond to a first group of additional molecules having values for the one or more molecular properties. In one or more examples, the gradient optimization technique can include moving to a different subset of the plurality of nodes of the latent space outside of the subset of the plurality of nodes and determining an additional group of second molecules representations that corresponds to the different subset of the plurality of nodes. The gradient optimization technique can also include determining, using the one or more second neural networks and based on the additional group of second molecules representations, where the additional third molecules representations can indicate additional third molecules. Further, the gradient optimization technique can include determining, using the one or more additional computational models and based on the additional third molecules, further values for the molecular property.

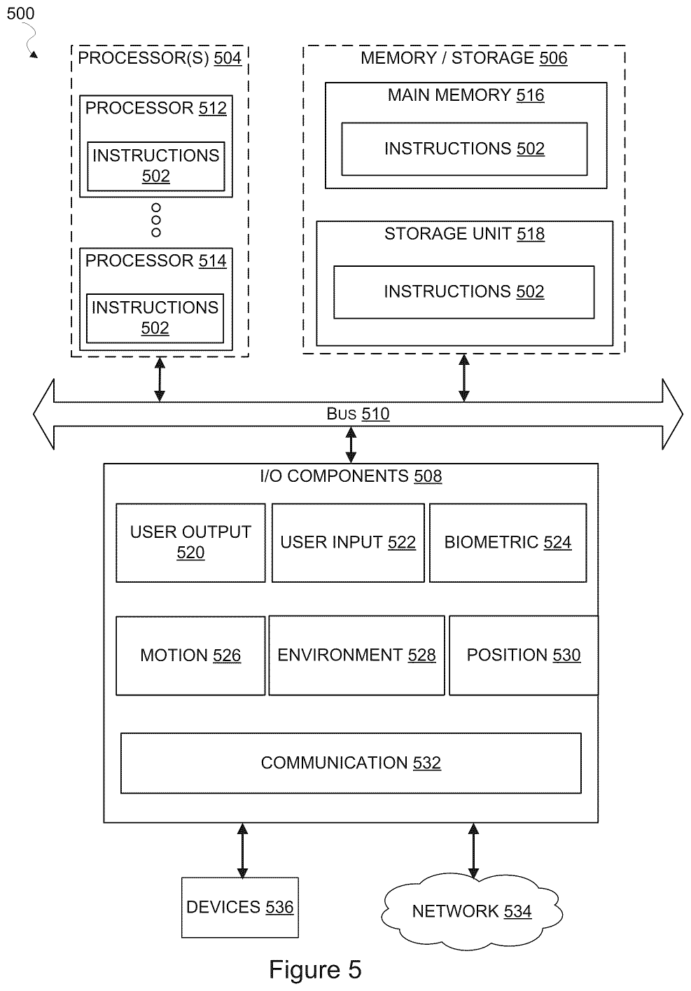

FIG. 5 is a block diagram illustrating components of a machine 500, according to some example implementations, able to read instructions from a machine-readable medium (e.g., a machine-readable storage medium) and perform any one or more of the methodologies discussed herein. Specifically, FIG. 5 shows a diagrammatic representation of the machine 500 in the example form of a computer system, within which instructions 502 (e.g., software, a program, an application, an applet, an app, or other executable code) for causing the machine 500 to perform any one or more of the methodologies discussed herein may be executed. As such, the instructions 502 may be used to implement modules or components described herein. The instructions 502 transform the general, non-programmed machine 500 into a particular machine 500 programmed to carry out the described and illustrated functions in the manner described. In alternative implementations, the machine 500 operates as a standalone device or may be coupled (e.g., networked) to other machines. In a networked deployment, the machine 500 may operate in the capacity of a server machine or a client machine in a server-client network environment, or as a peer machine in a peer-to-peer (or distributed) network environment. The machine 500 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 502, sequentially or otherwise, that specify actions to be taken by machine 500. Further, while only a single machine 500 is illustrated, the term “machine” shall also be taken to include a collection of machines that individually or jointly execute the instructions 502 to perform any one or more of the methodologies discussed herein.

The machine 500 may include processors 504, memory/storage 506, and I/O components 508, which may be configured to communicate with each other such as via a bus 510. In an example implementation, the processors 504 (e.g., a central processing unit (CPU), a reduced instruction set computing (RISC) processor, a complex instruction set computing (CISC) processor, a graphics processing unit (GPU), a digital signal processor (DSP), an application-specific integrated circuit (ASIC), a radio-frequency integrated circuit (RFIC), another processor, or any suitable combination thereof) may include, for example, a processor 512 and a processor 514 that may execute the instructions 502. The term “processor” is intended to include multi-core processors 504 that may comprise two or more independent processors (sometimes referred to as “cores”) that may execute instructions 502 contemporaneously. Although FIG. 5 shows multiple processors 504, the machine 500 may include a single processor 512 with a single core, a single processor 512 with multiple cores (e.g., a multi-core processor), multiple processors 512, 514 with a single core, multiple processors 512, 514 with multiple cores, or any combination thereof.

The memory/storage 506 may include memory, such as a main memory 516, or other memory storage, and a storage unit 518, both accessible to the processors 504 such as via the bus 510. The storage unit 518 and main memory 516 store the instructions 502 embodying any one or more of the methodologies or functions described herein. The instructions 502 may also reside, completely or partially, within the main memory 516, within the storage unit 518, within at least one of the processors 504 (e.g., within the processor's cache memory), or any suitable combination thereof, during execution thereof by the machine 500. Accordingly, the main memory 516, the storage unit 518, and the memory of processors 504 are examples of machine-readable media.

The I/O components 508 may include a wide variety of components to receive input, provide output, produce output, transmit information, exchange information, capture measurements, and so on. The specific I/O components 508 that are included in a particular machine 500 will depend on the type of machine. For example, portable machines such as mobile phones will likely include a touch input device or other such input mechanisms, while a headless server machine will likely not include such a touch input device. It will be appreciated that the I/O components 508 may include many other components that are not shown in FIG. 5. The I/O components 508 are grouped according to functionality merely for simplifying the following discussion and the grouping is in no way limiting. In various example implementations, the I/O components 508 may include user output components 520 and user input components 522. The user output components 520 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 522 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or other pointing instrument), tactile input components (e.g., a physical button, a touch screen that provides location or force of touches or touch gestures, or other tactile input components), audio input components (e.g., a microphone), and the like.

In further example implementations, the I/O components 508 may include biometric components 524, motion components 526, environmental components 528, or position components 530 among a wide array of other components. For example, the biometric components 524 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 526 may include acceleration sensor components (e.g., accelerometer), gravitation sensor components, rotation sensor components (e.g., gyroscope), and so forth. The environmental components 528 may include, for example, illumination sensor components (e.g., photometer), temperature sensor components (e.g., one or more thermometer that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 530 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 508 may include communication components 532 operable to couple the machine 500 to a network 534 or devices 536. For example, the communication components 532 may include a network interface component or other suitable device to interface with the network 534. In further examples, communication components 532 may include wired communication components, wireless communication components, cellular communication components, near field communication (NFC) components, Bluetooth® components (e.g., Bluetooth® Low Energy), Wi-Fi® components, and other communication components to provide communication via other modalities. The devices 536 may be another machine 500 or any of a wide variety of peripheral devices (e.g., a peripheral device coupled via a USB).

Moreover, the communication components 532 may detect identifiers or include components operable to detect identifiers. For example, the communication components 532 may include radio frequency identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 532, such as location via Internet Protocol (IP) geo-location, location via Wi-Fi® signal triangulation, location via detecting an NFC beacon signal that may indicate a particular location, and so forth.

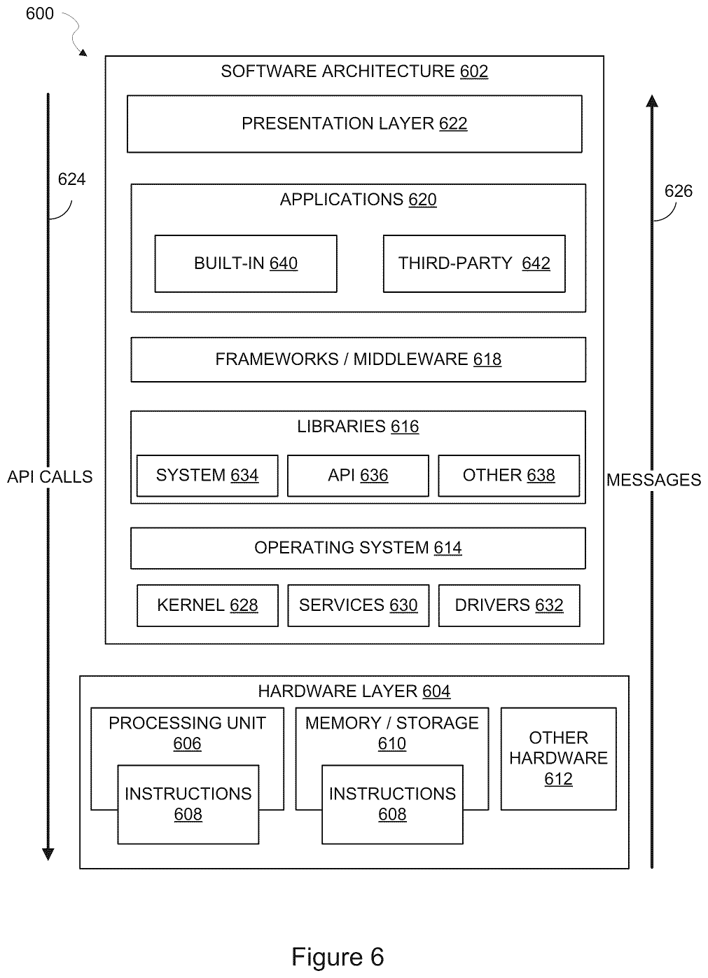

FIG. 6 is a block diagram illustrating system 600 that includes an example software architecture 602, which may be used in conjunction with various hardware architectures herein described. FIG. 6 is a non-limiting example of a software architecture, and it will be appreciated that many other architectures may be implemented to facilitate the functionality described herein. The software architecture 602 may execute on hardware such as machine 500 of FIG. 5 that includes, among other things, processors 504, memory/storage 506, and input/output (I/O) components 508. A representative hardware layer 604 is illustrated and can represent, for example, the machine 500 of FIG. 5. The representative hardware layer 604 includes a processing unit 606 having associated executable instructions 608. Executable instructions 608 represent the executable instructions of the software architecture 602, including implementation of the methods, components, and so forth described herein. The hardware layer 604 also includes at least one of memory or storage modules memory/storage 610, which also have executable instructions 608. The hardware layer 604 may also comprise other hardware 612.

In the example architecture of FIG. 6, the software architecture 602 may be conceptualized as a stack of layers where each layer provides particular functionality. For example, the software architecture 602 may include layers such as an operating system 614, libraries 616, frameworks/middleware 618, applications 620, and a presentation layer 622. Operationally, the applications 620 or other components within the layers may invoke API calls 624 through the software stack and receive messages 626 in response to the API calls 624. The layers illustrated are representative in nature and not all software architectures have all layers. For example, some mobile or special purpose operating systems may not provide a frameworks/middleware 618, while others may provide such a layer. Other software architectures may include additional or different layers.

The operating system 614 may manage hardware resources and provide common services. The operating system 614 may include, for example, a kernel 628, services 630, and drivers 632. The kernel 628 may act as an abstraction layer between the hardware and the other software layers. For example, the kernel 628 may be responsible for memory management, processor management (e.g., scheduling), component management, networking, security settings, and so on. The services 630 may provide other common services for the other software layers. The drivers 632 are responsible for controlling or interfacing with the underlying hardware. For instance, the drivers 632 include display drivers, camera drivers, Bluetooth® drivers, flash memory drivers, serial communication drivers (e.g., Universal Serial Bus (USB) drivers), Wi-Fi® drivers, audio drivers, power management drivers, and so forth depending on the hardware configuration.

The libraries 616 provide a common infrastructure that is used by at least one of the applications 620, other components, or layers. The libraries 616 provide functionality that allows other software components to perform tasks in an easier fashion than to interface directly with the underlying operating system 614 functionality (e.g., kernel 628, services 630, drivers 632). The libraries 616 may include system libraries 634 (e.g., C standard library) that may provide functions such as memory allocation functions, string manipulation functions, mathematical functions, and the like. In addition, the libraries 616 may include API libraries 636 such as media libraries (e.g., libraries to support presentation and manipulation of various media format such as MPEG4, H.264, MP3, AAC, AMR, JPG, PNG), graphics libraries (e.g., an OpenGL framework that may be used to render two-dimensional and three-dimensional in a graphic content on a display), database libraries (e.g., SQLite that may provide various relational database functions), web libraries (e.g., WebKit that may provide web browsing functionality), and the like. The libraries 616 may also include a wide variety of other libraries 638 to provide many other APIs to the applications 620 and other software components/modules.

The frameworks/middleware 618 (also sometimes referred to as middleware) provide a higher-level common infrastructure that may be used by the applications 620 or other software components/modules. For example, the frameworks/middleware 618 may provide various graphical user interface functions, high-level resource management, high-level location services, and so forth. The frameworks/middleware 618 may provide a broad spectrum of other APIs that may be utilized by the applications 620 or other software components/modules, some of which may be specific to a particular operating system 614 or platform.

The applications 620 include built-in applications 640 and third-party applications 642. Examples of representative built-in applications 640 may include, but are not limited to, a contacts application, a browser application, a book reader application, a location application, a media application, a messaging application, or a game application. Third-party applications 642 may include an application developed using the ANDROID™ or IOS™ software development kit (SDK) by an entity other than the vendor of the particular platform and may be mobile software running on a mobile operating system such as IOS™, ANDROID™, WINDOWS® Phone, or other mobile operating systems. The third-party applications 642 may invoke the API calls 624 provided by the mobile operating system (such as operating system 614) to facilitate functionality described herein.

The applications 620 may use built-in operating system functions (e.g., kernel 628, services 630, drivers 632), libraries 616, and frameworks/middleware 618 to create UIs to interact with users of the system. Alternatively, or additionally, in some systems, interactions with a user may occur through a presentation layer, such as presentation layer 622. In these systems, the application/component “logic” can be separated from the aspects of the application/component that interact with a user.

Unless otherwise indicated, all numbers expressing quantities of physical properties, chemical properties, dimensions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the implementations described herein. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

### Examples of the Disclosure

Generation of drug-like molecules with high binding affinity to target proteins remains a difficult and resource-intensive task in drug discovery. Existing approaches primarily employ reinforcement learning, Markov sampling, or deep generative models guided by Gaussian processes, which can be prohibitively slow when generating molecules with high binding affinity calculated by computationally expensive physics-based methods. We present Latent Inceptionism on Molecules (LIMO), which significantly accelerates molecule generation with an inceptionism-like technique. LIMO employs a variational autoencoder-generated latent space and property prediction by two neural networks in sequence to enable faster gradient-based reverse-optimization of molecular properties. Comprehensive experiments show that LIMO performs competitively on benchmark tasks and markedly outperforms state-of-the-art techniques on the novel task of generating drug-like compounds with high binding affinity, reaching nanomolar range against two protein targets. We corroborate these docking-based results with more accurate molecular dynamics-based calculations of absolute binding free energy and show that one of our generated drug-like compounds has a predicted K_{D} (a measure of binding affinity) of 6·10⁻¹⁴ M against the hu-man estrogen receptor, well beyond the affinities of typical early-stage drug candidates and most FDA-approved drugs to their respective targets.

Modern drug discovery is a long and expensive process, of-ten requiring billions of dollars and years of effort (Hughes et al., 2011). Accelerating the process and reducing its cost would have clear economic and human benefits. A central goal of the first stages of drug discovery, which comprise a significant fraction and cost of the entire drug discovery pipeline (Paul et al., 2010), is to find a compound that has high binding affinity to a designated protein target, while retaining favorable pharmacologic and chemical properties (Hughes et al., 2011). This task is difficult because there are on the order of 10; chemically feasible molecules in the drug-like size range (Polishchuk et al., 2013), and only a tiny fraction of these bind to any given target with an affinity high enough to make them candidate drugs. Currently, this is done with large experimental compound screens and iterative synthesis and testing by medicinal chemists.

Recently, deep generative models have been proposed to identify promising drug candidates (Guimaraes et al., 2017; Go\acute{m}ez-Bombarelli et al., 2018; Jin et al., 2018; Ma et al., 2018; You et al., 2018; Popova et al., 2019; Zhou et al., 2019; Jin et al., 2020b; Xie et al., 2021; Luo et al., 2021b), potentially circumventing much of the customary experimental work. However, even the best generative methods are prohibitively slow when optimizing for molecular properties that are computationally expensive to evaluate, such as binding affinity.

Here, we present a novel approach called Latent Inception-ism on Molecules (LIMO), a generative modeling frame-work for fast de novo molecule design that
builds on the variational autoencoder (VAE) frame-work, combined with a novel property predictor net-work architecture;
employs an inceptionism-like reverse optimization technique on a latent space to generate drug-like molecules with desirable properties;
is much faster than existing reinforcement learning-based methods (6-8× faster) and sampling-based approaches (12× faster), while maintaining or exceeding baseline performances on the generation of molecules with desired properties;
allows for the generation of molecules with desired properties while keeping a molecular substructure fixed, an important task in lead optimization;
markedly outperforms state-of-the-art methods in the novel task of generating drug-like molecules with high binding affinities to target proteins.

Domain state of the art. After a protein is identified as a potential drug target, a common drug discovery paradigm today involves performing an initial high-throughput experimental screening of available compounds to identify hit compounds, i.e., molecules that have some affinity to the target. Computational methods, such as docking (e.g. Santos-Martins et al. (2021); Friesner et al. (2004)) or more rigorous molecular dynamics-guided binding free energy calculations (Cournia et al., 2020) of compounds to a known 3D structure of the target protein can also play a role by prioritizing compounds for testing. Once hit compounds have been experimentally confirmed, they become starting points for the synthesis of chemically similar lead compounds that have improved activity but require further optimization (lead optimization) to become a drug candidate that is deemed promising enough to advance further through the drug discovery pipeline (Hughes et al., 2011). To accelerate this often years-long drug discovery stage, there is great interest in novel computational technologies.

An alternative to experimentally screening existing com-pounds is to design entirely novel compounds for synthesis and testing. This approach, termed de novo design, takes advantage of the target protein's 3D structure. Genetic algorithms (e.g., Spiegel & Durrant (2020)) and rule-based approaches (e.g. Allen et al. (2017)) have been developed for this task. However, these techniques are often slow and tend to be too rigid to be fully integrated into the drug discovery process, where many molecular properties and synthesizability must be considered simultaneously. For example, AutoGrow4 (Spiegel & Durrant, 2020), a state-of-the-art genetic algorithm, produces molecules with high binding affinities, but can also lead to toxic moieties and excessive molecular weights, while also being limited in the molecular space available for exploration. In contrast, recent machine learning methods offer greater flexibility and hence new promise for de novo drug design (Carracedo-Reboredo et al., 2021), as summarized below.

Generative models for molecule design. Deep generative models use a learned latent space to represent the distribution of drug-like molecules. Early work (Go\acute{m}ez-Bombarelli et al., 2018) applies a variational autoencoder (VAE, Kingma & Welling (2013)) to map SMILES strings (Weininger, 1988) to a continuous latent space. But SMILES-based representations struggle with generating both syntactically and semantically valid strings. Other works address this limitation by incorporating rules into the VAE decoder to only generate valid molecules (Kusner-et al., 2017, Dai et al., 2018). Junction tree VAEs (Jin et al., 2018; 2019) use a scaffold junction tree to assemble building blocks into an always-valid molecular graph, and have been improved with RL-like sampling and optimization techniques (Tripp et al., 2020; Notin et al., 2021). DruGAN (Kadurin et al., 2017) further extends VAEs to an implicit GAN-based generative model. OptiMol uses a VAE to output molecular strings, but takes molecular graphs as input (Boitreaud et al., 2020). Shen et al. (2021) forego a latent space altogether and assemble symbols directly.

Apart from sequence generation, graph generative models have also been proposed (Ma et al., 2018; Simonovsky & Komodakis, 2018; De Cao & Kipf, 2018; Li et al., 2018; Fu et al., 2022; Zang & Wang, 2020; Luo et al., 2021b; Jin et al., 2020a; Luo et al., 2021a). As generative models do not directly control molecular properties, existing methods often use a surrogate model (Gaussian process or neural network) to predict molecular properties from the latent space, and guide optimization on the latent space toward molecules with desired properties (e.g., log P, QED, binding affinity). For example, MoFlow (Zang & Wang, 2020) predicts molecular properties from a latent space using a neural network but has difficulty generating molecules with high property scores. Instead, we propose the prediction of properties from the decoded molecular space, which appears to greatly increase the property scores of generated molecules. Xie et al. (2021) propose Monte Carlo sampling to explore molecular space and Nigam et al. (2020) propose a genetic algorithm with a neural network-based discriminator, both of which require an extremely large number of calls to property functions and therefore are less useful when optimizing complex, expensive-to-evaluate property functions.

In general, generative models are very fast in generating molecules. However, as current generative models cannot effectively find molecules in their latent spaces that have desired properties, they have so far been outperformed by reinforcement learning-based methods that directly optimize molecules for desired properties.

Reinforcement learning-based molecule generation. Reinforcement learning (RL) methods directly optimize molecular properties by systematically constructing or altering a molecular graph (You et al., 2018; Zhou et al., 2019; Jin et al., 2020b; Guimaraes et al., 2017; Popova et al., 2019; De Cao & Kipf, 2018; Zhavoronkov et al., 2019; Olivecrona et al., 2017; Shi et al., 2020; Luo et al., 2021b; Jeon & Kim, 2020). These methods appear to be the most powerful at generating molecules with desired properties but are slow and require many calls to the property estimation function. This is problematic when applying RL to computationally expensive but highly useful property functions like physics-based (e.g., docking) computed binding affinity, rather than simple, easily computed measures such as log P. RationaleRL (Jin et al., 2020b) theoretically avoids the need to sample a large number of molecules by collecting “rationales” from existing molecules with desired properties, and combining them into molecules with multiple desired properties, but by design this method is not applicable to de novo drug discovery.

Methodology. We present Latent Inceptionism on Molecules (LIMO), a molecular generation framework. We use a VAE to learn a real-valued latent space representation of the drug-like chemical space. However, contrary to previous work, we use two neural networks (a decoder and predictor) in sequence to perform inceptionism-like reverse optimization of molecular properties on the latent space.

We use a decoder network to generate an intermediate real-valued molecular representation to improve the prediction, and therefore optimization, of molecular properties while keeping the prediction differentiable, allowing the use of efficient gradient-based optimizers. We use self-referencing embedded strings (SELFIES, Krenn et al. (2020)) to ensure chemical validity during optimization. With these novelties, LIMO is able to achieve performance on par with reinforcement learning methods while being orders of magnitude faster. On the highly useful task of structure-based computed binding affinity optimization, LIMO markedly outperforms state-of-the-art (including RL) methods, while also being much faster.

Variational Autoencoder. Define a string representation of a molecule x=(x₁, . . . , xₙ). Each xᵢ takes its value from a set of d possible symbols S={s₁, . . . , s_{d}}, where each symbol is one component of a self-referencing embedded string (SELFIES) defining a molecule (Krenn et al., 2020). We aim to produce n independent distributions y=(y₁, . . . , yₙ), where each yᵢ∈[0, 1]^{d} is the parameter for a multinomial distribution over the d symbols in S. The output string \hat{x}=(\hat{x}₁, . . . , \hat{x}ₙ) is obtained from y by selecting the symbol with the highest probability:

\hat{x}ᵢ=s_{d}*,dᵢ=argmax_{d}{y_{i,1}, . . . ,y_{i,d}} (1).

All SELFIES strings correspond to valid molecules, allowing us to transform the continuous-value probabilities y into an always-valid discrete molecule.

We train a VAE (Kingma & Welling, 2013) to encode x to a latent space z∈R^{m} of dimensionality m and decode to y. We optimize the VAE using the evidence lower bound (ELBO) loss function. Each input symbol in the representation string passes through an embedding layer, and then two fully-connected networks (the encoder and decoder). Reconstruction loss is calculated using the negative log-likelihood over a one-hot encoded representation of the input molecule. Once trained, the VAE can generate novel and drug-like molecules, with similar molecules lying next to each other in the latent space. To generate random molecules, we sample from the latent space z using N (0ₘ, Iₘ), and decode it into a string representation.

Property Predictor. We employ a separate network to predict molecular properties. While earlier works train the VAE and property predictor jointly (Jin et al., 2018; Go\acute{m}ez-Bombarelli et al., 2018), we train the property predictor after the VAE has been fully trained (i.e. we freeze the VAE weights) for three reasons: firstly, generative modeling requires significantly more molecular data than the regression task of predicting molecular properties. There is no need to acquire property data for all the molecules used by the generative model. This is especially relevant when such data is expensive to obtain, e.g., docking-based binding affinity that takes seconds to calculate per molecule. Secondly, the trained generative model allows us to query the ground-truth molecular property function with its generated molecules, giving an informative and diverse training set for property prediction. Thirdly, adding new properties under this training scheme does not require retraining of the VAE, only of the property predictor, which is much more efficient.

Crucially, we introduce a novel architecture consisting of stacking the VAE decoder and the property predictor. The property predictor uses the output of the VAE decoder as its input, as opposed to predicting properties directly from the latent space like previous works (e.g. Jin et al. (2018); Go\acute{m}ez-Bombarelli et al. (2018); Zang & Wang (2020)). The intuition is that the map from molecular space to property is easier to learn than that from the latent space to property. We later present results confirming this intuition, both in terms of prediction accuracy and overall optimization ability, suggesting that the proposed stacking improves optimization by allowing more accurate prediction of molecular properties through a more direct molecular representation. Using such an intermediate molecular representation from the VAE decoder also allows us to fix a substructure of the generated molecule, giving LIMO the ability to perform the unique, compared to many other VAE-based architectures, ability to perform substructure-constrained optimization.

Define the VAE encoder f_{enc}: x 1→z and decoder f_{dec}: z 1→\hat{x}, a property prediction network g_{θ}: \hat{x} 1→R with parameters θ, and a ground-truth property estimation function π: \hat{x} 1→R that computes a molecular property such as log P or binding affinity. We first generate examples to train g_{θ} by sampling random molecules from the latent space z using a normal distribution N (0ₘ, Iₘ). Then, we optimize the parameters θ of the property predictor by minimizing the mean square error (MSE) of predicted properties

[symbol]

₀(θ)=∥g_{θ}(f_{dec}(z))−(π(f_{dec}(z))∥² (2)

Reverse Optimization. After training, we freeze the weights of f_{dec} and g_{θ}, and make z trainable to optimize the latent space toward locations that decode to molecules with desirable properties. This is a similar technique to inceptionism, which involves backpropagating from the output of a network to its input so that the input is altered to affect the output in a desired way (Mordvintsev et al., 2015).

To maximize properties, given a set of k property predictors (g¹, . . . , g^{k}) and weights (w₁, . . . , wₖ), we minimize the following function using the Adam optimizer, initialized from z˜N (0ₘ, Iₘ):

[symbol]

₁(z)=−Σᵢ₌₁^{k}wᵢ·gⁱ(f_{dec}(z)) (3)

Crucially, since both f_{dec} and g are neural networks, gradient-based techniques can be used for efficient optimization of z. The weights (w₁, . . . , wₖ) are hyperparameters determined by a random grid search.

In lead optimization, a common task is to generate molecules with desired properties while keeping a given substructure of the molecules fixed. To apply reverse optimization to this task, we define a mask M∈{0, 1}^{n×d}, where M_{i,j} corresponds to the SELFIES symbol of index j at position i in a molecular string. We assign M_{i,j}=1 where the desired substructure is present and the corresponding symbol cannot be changed, 0 otherwise. For an optimization starting point, we then reconstruct a molecule xₛₜₐᵣₜ that has the desired substructure: \hat{x}ₛₜₐᵣₜ=f_{dec}(f_{enc}(xₛₜₐᵣₜ)). To optimize z while also keeping a substructure constant, we add an additional loss £₂ to the £₁ used in Equation 3:

[symbol]

₂(Z)=λΣᵢ₌₁ⁿΣⱼ₌₁^{d}(M_{i,j}·(f_{dec}(z)_{i,j}−(\hat{x}ₛₜₐᵣₜ)_{i,j}))² (4),

where λ is a weighting term we set to 1,000.

Refinement. Filtering. Following multi-objective optimization, we per-form a filtering step to exclude non-drug-like molecules. Using the distributions of quantitative estimate of drug-likeness (QED) and synthetic accessibility (SA) scores on drug-like datasets (Bickerton et al., 2012; Ertl & Schuffenhauer, 2009), we define cutoff values reasonably within the range of currently marketed drugs. Molecules not reaching these cutoffs are excluded from consideration. We also exclude molecules with either too small or too large chemical cycles (rings), as these are usually difficult to synthesize but are not excluded effectively by the SA metric. Specifically, we exclude molecules not satisfying (QED>0.4)∧(SA<5.5)∧(no rings with <5 or >6 atoms).

Fine-tuning. For some tasks, we observe that LIMO is effective in generating molecules with reasonably high property scores that could nonetheless be improved slightly by small, atom-level changes. To do this, we performed a greedy local search around the chemical space of a generated molecule by systematically replacing carbons with heteroatoms and retaining changes that lead to the most improvement. The algorithm is detailed in Appendix A.3.

Experiments. We apply LIMO to QED and penalized log P (p-log P) maximization, log P targeting (You et al., 2018), similarity-constrained p-log P maximization (Jin et al., 2018), substructure-constrained log P extremization, and single and multi-objective binding affinity maximization. All of these tasks are typical challenges in drug discovery, especially optimization around a substructure and maximization of binding affinity. See Appendix A.1 for description of each task, and Appendix B.1 for results from the random generation of molecules.

Experimental Setup. Dataset. For all optimization tasks, we use the benchmark ZINC250k dataset, which contains≈250,000 purchasable, drug-like molecules (Irwin et al., 2012). We use AutoDock-GPU (Santos-Martins et al., 2021) to compute binding affinity, as described in Appendix A.6, and RDKit to compute other molecular properties. For the random generation task, we train on the ZINC-based≈2 million molecule MOSES dataset (Polykovskiy et al., 2020).

Model training. All experiments use identical autoencoder weights and a latent space dimension of 1024. We select hyperparameters using a random grid search. The property predictor is trained independently for each of the following properties: log P (octanol-water partition coefficient), p-log P (Jin et al., 2018), SA (Ertl & Schuffenhauer, 2009), QED (Bickerton et al., 2012), and binding affinity to two targets (calculated by AutoDock-GPU, Santos-Martins et al. (2021)). 100 k training examples were used for all properties except binding affinity, where 10 k were used due to speed concerns. See Appendix A.5 for model training details.

Baselines. We compare with the following state-of-the-art molecular design baselines: JT-VAE (Jin et al., 2018), GCPN (You et al., 2018), MolDQN (Zhou et al., 2019), MARS (Xie et al., 2021), and GraphDF (Luo et al., 2021b). Each technique is described in Appendix 5.

Protein targets. For tasks involving binding affinity optimization, we target the binding sites of two human proteins.

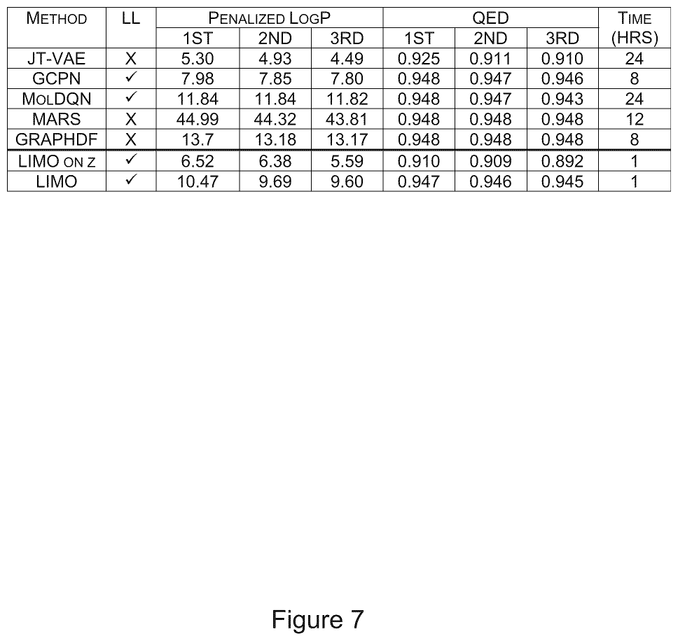

QED and Penalized log P Maximization. FIG. 7 includes a table showing results of LIMO and baselines on the generation of molecules with high penalized log P and QED scores. For both properties, we report the top 3 scores of 100 k generated molecules, as well as the total time (generation+testing) taken by each method. As an ablative study, we apply LIMO with property prediction directly on the latent space (“LIMO on z”) as opposed to regular LIMO, which performs property prediction on the decoded molecule \hat{x} (see Section 3.2). Both methods underwent the same hyperparameter tuning as described in Appendix A.5. We see that the extra novel step of decoding the latent space and then performing property prediction offers a significant advantage for the optimization of molecules. To elucidate this improvement, an unseen test set of 1,000 molecules was generated using the VAE and used to test the prediction performance of the property predictor. We observe an r²=0.04 between real and predicted properties for “LIMO on z”, and r²=0.38 for LIMO. This large predictive performance boost explains the observed improvements in the optimization of molecules, as the model is better able to generalize what makes a molecule bind well. We also replaced LIMO's fully-connected VAE encoder and decoder each with an 8-layer, 512 hidden dimension LSTM and found significantly worse performance, e.g., a maximum QED score of 0.3. The addition of a self-attention layer after the LS™ encoder did not significantly improve performance.

We observe that LIMO achieves competitive results among deep generative and RL-based models (i.e., all methods except MARS) while taking significantly less time. Note that p-log P is a “broken” metric that is almost entirely dependent on molecule length (Zhou et al., 2019). Without a length limit, MARS can easily generate long carbon chains with high p-log P. Among models with a molecule length limit (GCPN, MolDQN, and LIMO), LIMO generates molecules with p-log P similar to MolDQN, the strongest baseline. Similarly, QED suffers from boundary effects around its maximum score of 0.948 (Zhou et al., 2019), which LIMO gets very close to. Drugs with a QED score above 0.9 are very rare (Bickerton et al., 2012), so achieving close to this maximum score is sufficient for drug discovery purposes.

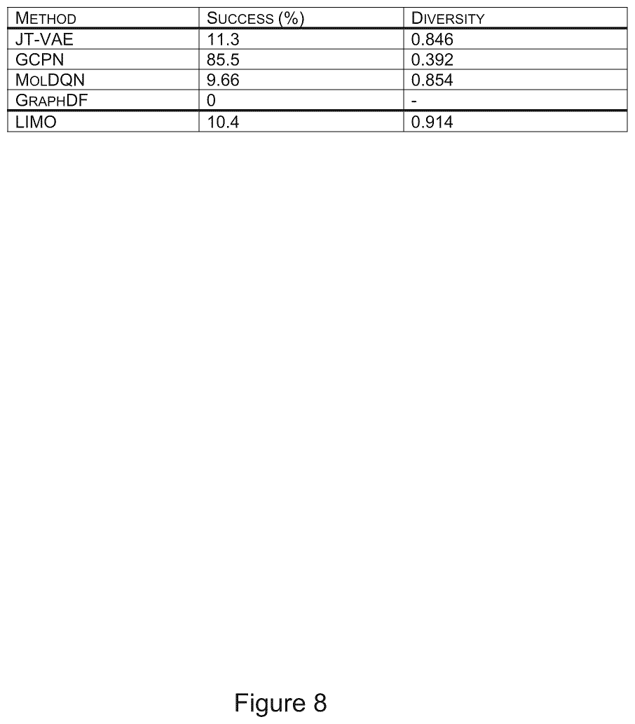

log P Targeting. FIG. 8 includes a table that reports on the ability of LIMO to generate molecules with log P targeted to the range −2.5<log P<−2.0. LIMO achieves the highest diversity among generated molecules within the targeted log P range, and, although it has a lower success rate than other methods, it generates 33 molecules per second within the target range. This is similar to the overall generation speed of other models, but due to a lack of available code for this task, we were not able to compare exact speeds.

Similarity-constrained Penalized log P Maximization. Following the procedures described for JT-VAE (Jin et al., 2018), we select the 800 molecules with the lowest p-log P scores in the ZINC250k dataset and aim to generate new molecules with a higher p-log P yet similarity to the original molecule. Similarity is measured by Tanimoto similarity between Morgan fingerprints with a cutoff value δ. Each of the 800 starting molecules are encoded into the latent space using the VAE encoder, 1,000 gradient ascent steps (Section 3.3) are completed for each, then the generated molecules out of all gradient ascent steps with the highest p-log P that satisfy the similarity constraint are chosen.

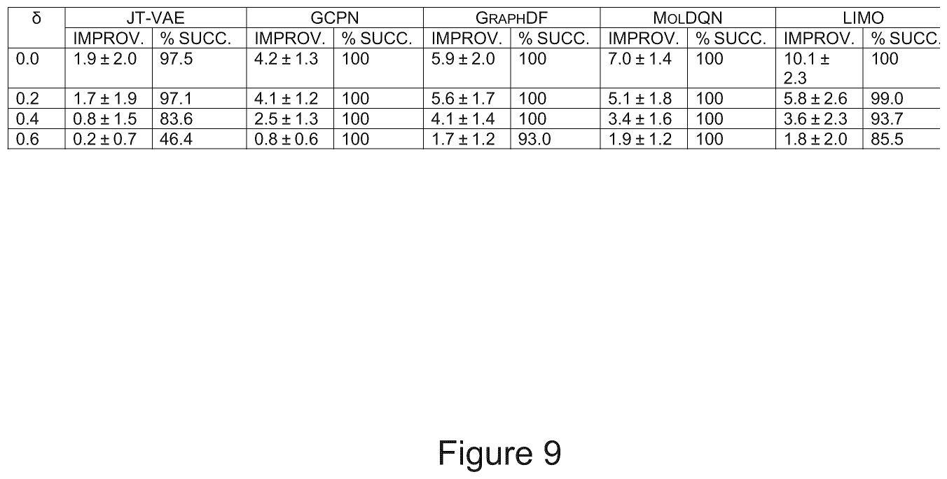

Results for the similarity-constrained p-log P maximization task are summarized in the table shown in FIG. 9. For the two lowest similarity constraints (δ=0.0, 0.2), LIMO achieves the highest penalized log P improvement, while its improvement is statistically indistinguishable from other methods at higher values of δ. This shows the power of LIMO for unconstrained optimization, and the ability to reach competitive performance in more constrained settings.

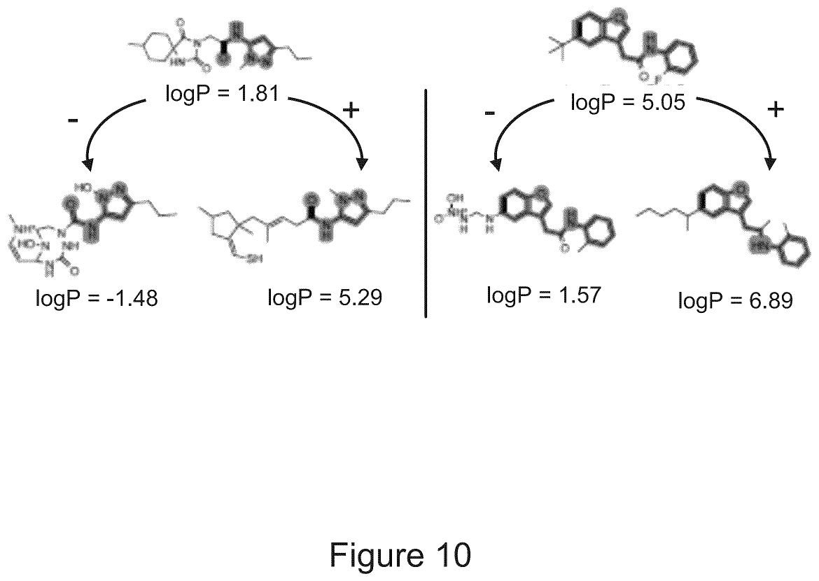

Results for the substructure-constrained log P extremization task are shown in FIG. 10. We chose two molecules from ZINC250k to act as starting molecules and defined the substructures of these starting molecules to be fixed, then performed both maximization and minimization of log P using LIMO, as described in Equation 4. As illustrated, we can successfully increase or decrease log P as desired while keeping the substructure constant in both cases.

This task is common during the lead optimization stage of drug development, where a synthetic pathway to reach an exact substructure with proven activity is established, but molecular groups around this substructure are more malleable and have not yet been determined. This is not captured in the similarity-constrained optimization task above, which uses more general whole-molecule similarity metrics.

While previous works address the challenge of property optimization around a fixed substructure (Hataya et al., 2021; Lim et al., 2020; Maziarz et al., 2021), LIMO is one of the few VAE-based methods that can easily perform such optimization. Thanks to its unique decoding step of generating an intermediate molecular string prior to property prediction, LIMO brings the speed benefits of VAE techniques to the substructure optimization task.

Single-objective Binding Affinity Maximization. Producing molecules with high binding affinity to the target protein is the primary goal of early drug discovery (Hughes et al., 2011), and its optimization using a docking-based binding affinity estimator, which is especially powerful in the de noto setting, is relatively novel to the ML-based molecule generation literature. Many previous approaches have attempted to optimize affinity by leveraging knowledge of existing binders (e.g. Zhavoronkov et al. (2019): Jeon & Kim (2020); Luo et al. (2021a)), but they often lack generalizability to targets without such binders. Therefore, we focus on molecule optimization in the de novo setting through the use of a docking-based affinity estimator.

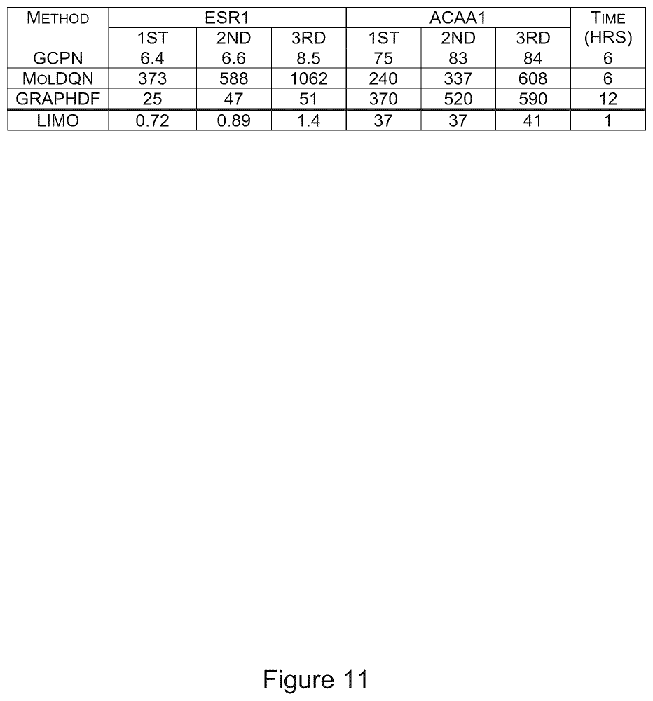

We target the binding sites of two human proteins: estro-gen receptor (PDB ESR1, UniProt P03372) and peroxisomal acetyl-CoA acyl transferase 1 (PDB ACAA1, UniProt P09110) (see Section 4.1 for details). For both of our protein targets we report the top 3 highest affinities (i.e., lowest dis-sociation constants, K), as estimated with AutoDockGPU) of 10 k total generated molecules from each method. As shown in the table of FIG. 11, LIMO generates compounds with higher computed binding affinities in far less time than prior state-of-the-art methods. We chose GCPN, MolDQN, GraphDF, and MARS as baseline comparisons because of their strong performance on other single-objective optimization tasks.

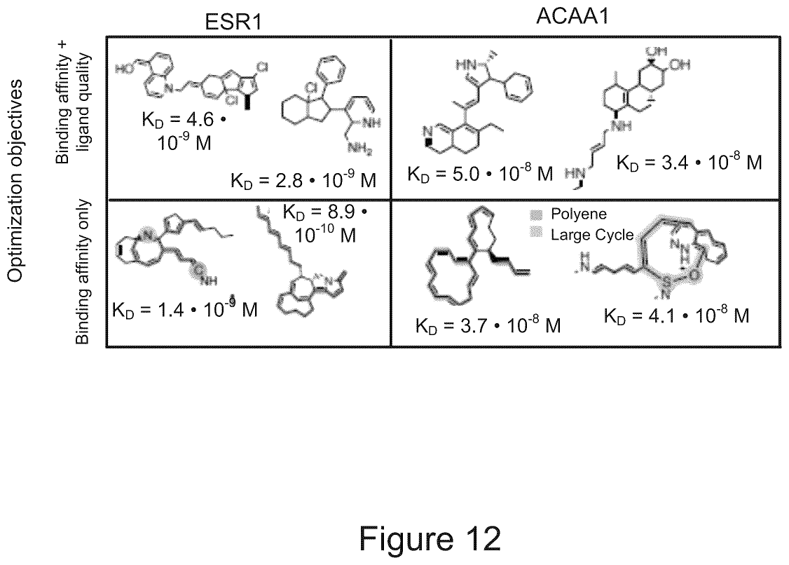

The chemical structures of two molecules generated by LIMO when only optimizing for binding affinity are shown in the bottom row of FIG. 12 for both protein targets. While these molecules have relatively high affinities, they would have little utility in drug discovery because they are pharmacologically and synthetically problematic. For example, we highlight two major moieties, polyenes and large (>8 atoms) cycles, that are regarded by domain experts as highly problematic due to reactivity/toxicity and synthesizability concerns, respectively (see Birch & Sibley (2017); Hussain et al. (2014); Abdelraheem et al. (2016)). Molecules generated from GCPN, MolDQN, GraphDF, and MARS had similar issues. These moieties are large structural issues that cannot be fixed with small tweaks following optimization, so we added measures of ligand quality into our optimization process as detailed in the following subsection.

Multi-objective Binding Affinity Maximization. To generate molecules with high computed binding affinity and pharmacologic and synthetic desirability, we simultaneously optimize molecules for computed binding affinity, drug-likeness (QED), and synthesizability (SA) scores. Distributions of properties before and after multi-objective optimization are shown in Appendix B.2. For each protein target, we generate 100 k molecules, then apply the two refinement steps described in Section 3.4. We selected the two compounds with the highest affinity from this process for each protein target, which are shown in the top row of FIG. 12. These compounds are more drug-like and synthetically accessible than those generated by single-objective optimization (FIG. 12, bottom row), but still have high predicted binding affinities (i.e., low K_{D} values), making them promising drug candidates. We analyze and compare these compounds in the subsequent section.

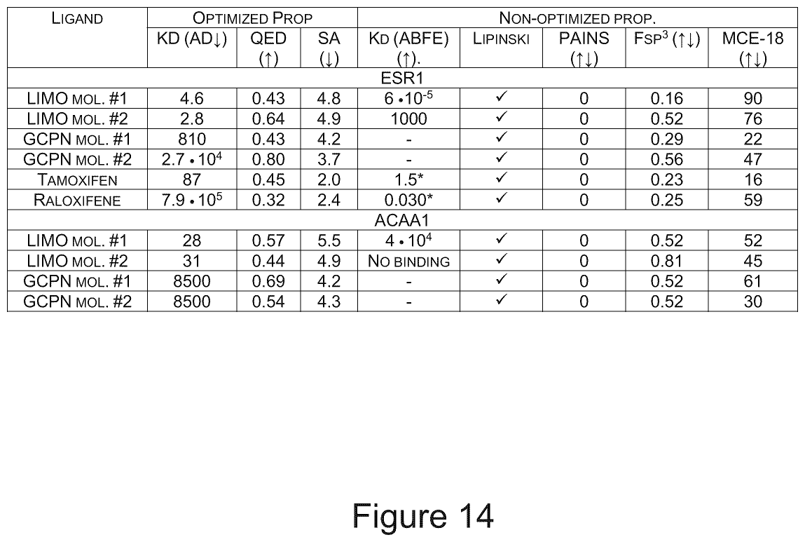

Compound analysis. FIG. 14 includes a table showing the binding and drug-likeness metrics of two generated compounds for both ESR1 and ACAA1 (the same as those shown in the top row of FIG. 3). For ESR1, we compare our compounds to tamoxifen and raloxifene, two modern breast cancer drugs on the market that target this protein. We also compare with com-pounds generated by GCPN, the second strongest method behind LIMO for single-objective binding affinity maximization, with identical multi-objective weights and the same filtering step as LIMO. For each compound, we report the metrics described in the Appendix A.2. The first three metrics given are “Optimized properties” that are explicitly optimized for, while the others are not used in optimization but are still useful for compound evaluation.

LIMO significantly outperforms GCPN, which generates molecules with such low computed affinity (high K_{D}) as to be of relatively low utility in drug discovery, regardless of drug-likeness or synthesizability metrics, because they are unlikely to bind their targets at all.

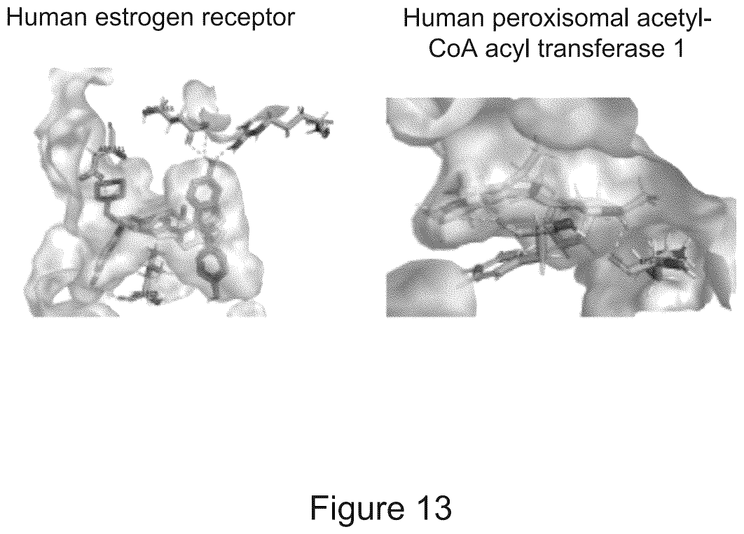

Visualization and corroboration of binding affinities. To confirm that the ligands generated by LIMO are likely to bind their target proteins with high affinity and do not score well due to inaccuracies or shortcuts used in the AutoDock-GPU scoring function, we visualized their docked poses in 3D to look for physically reasonable bound conformations and energetically favorable ligand-protein interactions. The 3D binding poses produced by the docking software for one of the two generated ligands for each protein (FIG. 13) show that they fit well into the protein binding pocket and promote favorable ligand-protein interactions.

We furthermore ran detailed, molecular dynamics-based, absolute binding free energy calculations (ABFE, Appendix A.7) (Gilson et al., 1997; Cournia et al., 2020) to obtain more reliable estimates of the affinities of LIMO-generated compounds for their targeted proteins than the predictions from docking. As shown in Table 5, LIMO generated an ESR1 ligand with an ABFE-predicted dissociation constant (K_{D}) of 6·10⁻⁵ nM, much better than typical K_{D} values of e.g. 1000 nM obtained from experimental compound screening and better even than the K_{D} values of tamoxifen and raloxifene, two drugs that bind ESR1 with high affinity. The LIMO compounds even exceed these drugs on many drug-likeness metrics. Without experimental confirmation, we cannot be sure these molecules bind so well, but the results from these state-of-the-art calculations are encouraging.

Discussion and Conclusions. We present LIMO, a generative modeling framework for de novo molecule design. LIMO utilizes a VAE latent space and two neural networks in sequence to reverse-optimize molecular properties, allowing the use of efficient gradient-based optimizers to achieve competitive results on benchmark tasks in significantly less time. On the task of generating molecules with high binding affinity, a novel task in the ML-based molecule generation literature, LIMO outperforms all state-of-the-art baselines.

LIMO promises multiple applications in drug discovery. The ability to quickly generate high-affinity compounds can accelerate target validation with biological probes that can be used to confirm the proposed biological effect of a target. LIMO also has the potential to accelerate lead generation and optimization by jumping directly to drug-like, synthesizable, high affinity compounds, thus bypassing the traditional hit identification, hit-to-lead, and lead optimization steps. While “unconstrained” LIMO can quickly generate high-affinity compounds, it has the additional ability to perform substructure-constrained property optimization, which is especially useful during the lead optimization stage where one has an established substructure with a synthetic pathway and wishes to “decorate” around it for improved activity or pharmacologic properties.

While LIMO can generate very high affinity compounds as computed by docking software, as is its goal, the utility of compounds only vetted by docking software may be questioned. As shown in FIG. 14, AutoDock-GPU computed binding affinities do not correlate very well with more accurate ABFE results. This is a well-known result (Cournia et al, 2020), but we believe having docking-predicted high affinity compounds is still of relatively high utility in drug discovery, even if some (or most) generated compounds are “false positives.” As LIMO can generate hundreds of diverse docking-computed nanomolar range compounds against a target in hours, it is likely that some of those compounds will actually bind a target well. This is a unique advantage of LIMO, as it is able to generate many candidate compounds very quickly, allowing for aggressive filtering downstream. Indeed, we have generated a highly favorable compound (K_{D}=6·10⁻¹⁴ M) as calculated by ABFE, even more favorable than AutoDock-GPU predictions, out of only two generated candidates. The addition of further automated binding affinity confirmation into the LIMO pipeline, e.g., with additional docking software or automated ABFE calculation, is a promising direction for future work. Other future directions include exploring the use of different molecular representation and model architectures in LIMO, the use of better optimizers beyond simple gradient-based methods, and the application of LIMO to more or multiple simultaneous protein targets.

Experiment Description and Baselines

Tasks

Random generation of molecules: Generate random molecules by sampling from the latent space of the generative model. As later optimization relies on these generated molecules as starting points, it is important that they be novel, diverse, and unique.

QED and penalized log P maximization: Generate molecules with high penalized log P (p-log P, estimated octanol-water partition coefficient penalized by synthetic accessibility (SA) score and number of cycles with more than six atoms (Jin et al., 2018)) and quantitative estimate of drug-likeness (QED, (Bickerton et al., 2012)) scores. These properties are important considerations in drug discovery, and this task shows the ability of a model to optimize salient aspects of a molecule, even if maximization of these properties by themselves is of low utility (Zhou et al., 2019).

log P targeting: Generate molecules with log P within a specified range. In drug discovery, a log P within a given range is often taken as an approximate indicator that a molecule will have favorable pharmacokinetic properties.

Similarity-constrained penalized log P maximization: For each molecule in a set of starting molecules, generate a novel molecule with a high penalized log P (p-log P) score while retaining similarity (as defined by Tanimoto similarity between Morgan fingerprints, Rogers & Hahn (2010)) to the original molecule. This mimics the drug discovery task of adjustment of an active starting molecule's log P while keeping similarity to the starting molecule to retain biological activity.

Substructure-constrained log P extremization: Generate molecules with either high or low log P scores while keeping a subgraph of a starting molecule fixed. This task mimics the drug discovery goal of optimizing around (“decorating”) an existing substructure to fine-tune activity or adjust pharmacologic properties. This is common in the lead optimization stage of drug development, where a synthetic pathway to reach an exact substructure with proven activity is established, but molecular groups around this substructure are more malleable and not yet established. This task is not captured in the similarity-constrained optimization task above, which uses more general whole-molecule similarity metrics.

Single-objective binding affinity maximization: Generate molecules with high computed binding affinity for two protein targets as determined by docking software. Reaching high binding affinities is the primary goal of early drug discovery, and its optimization using a physics-based affinity estimator is a relatively novel task in the ML-based molecule generation literature. Previous attempts to optimize affinity have relied on knowledge of existing binders (Zhavoronkov et al., 2019; Jeon & Kim, 2020; Luo et al., 2021a), which lacks the generalizability of physics-based estimators to targets without known binders.

Multi-objective binding affinity maximization: Generate molecules with favorable computed binding affinity, QED, and SA scores. This task has high utility in drug discovery, as it addresses targeting, pharmacokinetic properties, and ease of synthesis. Development of molecules satisfying all these considerations is challenging, and to the best of our knowledge, is a novel task in the ML-based molecule generation literature.

Protein targets. We target the binding sites of two human proteins:
i. Human estrogen receptor (ESR1): This well-characterized protein is a target of drugs used to treat breast cancer. It was chosen for its disease relevance and its many known binders, which are good points of comparison with generated molecules. Although known binders exist, LIMO was not fed any information beyond a crystal structure of the protein (PDB 1ERR) used for docking calculations and the location of the binding site.
ii. Human peroxisomal acetyl-CoA acyl transferase 1 (ACAA1): This enzyme has no known binders but does have a crystal structure (PDB 211K) with a potential drug-binding pocket, which we target to show the ability of LIMO for de novo drug design. We found this protein with the help of the Structural Genomics Consortium, which highlighted this protein as a potentially disease-relevant target with a known crystal structure, but no known binders.

Molecule metrics. We report the following metrics for our multi-objective optimized molecules, all of which are given by ADMETLab 2.0 (Xiong et al., 2021) except binding affinities:
i. K_{D} (AutoDock-GPU): Dissociation constant K_{D} in nanomolar, as calculated by AutoDock-GPU. Lower K_{D} is associated with better binding (i.e., higher affinity) (Santos-Martins et al., 2021)
ii. K_{D} (ABFE): Dissociation constant K_{D} in nanomolar, as calculated by absolute binding free energy (ABFE) calculations, which are generally more accurate than AutoDock-GPU scores (Cournia et al., 2020)
iii. QED: Quantitative estimate of drug-likeness score, higher is better (Bickerton et al., 2012)
iv. SA: Synthetic accessibility score, lower is better (Ertl & Schuffenhauer, 2009)
v. Lipinksi: Lipinski's rule of 5 is a commonly used rule of thumb for drug-likeness (Lipinski et al., 2001). Compounds that pass all or all but one of four components are considered more likely to be suitable as drugs.
vi. PAINS: Number of PAINS alerts. These alerts detect compounds likely to have non-specific activity against a wide array of biological targets, making them undesirable as drugs. Lower is better (Baell & Holloway, 2010
vii. Fsp³: The fraction of sp3 hybridized carbons, which is thought to correlate with favorable drug properties. Higher is better (Wei et al., 2020)
viii. MCE-18: A measure of molecular novelty based on complexity measures. Higher is better (Ivanenkov et al., 2019)

Fine Tuning Algorithm. Algorithm 1 Molecule fine-tuning algorithm. Require: The starting molecule M to be optimized and a function π(m) that calculates a property for molecule m

```
R ← {N, O, Cl, F}
bestProperty ← π(M)
while bestProperty is improving do
  bestMolecule = M
  for all carbon atoms in M not adjacent to any atoms ∈ R do
    for all potential replacement atoms ∈ R do
      m ← M with the carbon atom replaced
      if m is valid and π(m) is better than π(bestMolecule) then
          bestMolecule ← m
        end if
      end for
    end for
  M ← bestMolecule
end while
```

Baselines. We compare with the following baselines:
i. JT-VAE (Jin et al., 2018): a VAE-based generative model that first generates a scaffold junction tree and then assembles nodes in the tree into a molecular graph.
ii. GCPN (You et al., 2018): an RL agent that successively constructs a molecule by optimizing a reward composed of molecular property objectives and adversarial loss. For running baselines, we use code from https://github.com/bowenliul6/rl_graph_generation.
iii. MolDQN (Zhou et al., 2019): an RL framework that uses chemical domain knowledge and double Q-learning. For running baselines, we use code from https://github.com/aksub99/MolDQN-pytorch.
iv. MARS (Xie et al., 2021): a sampling method based on Markov chain Monte Carlo that uses an adaptive fragment-editing proposal distribution based on GNN.
v. GraphDF (Luo et al., 2021b): a normalizing flow model for graph generation that uses a discrete latent variable model, fine-tuned with RL. For running baselines, we use code from https://github.com/divelab/DIG.

To generate results from baselines, we ran each method until little improvement was observed. For methods without an explicit generation process (i.e., GCPN, MolDQN, and MARS), we took the highest property scores from all molecules generated. For methods with an explicit generation process (GraphDF), we trained until little improvement was observed and then sampled the same number of molecules as was sampled from LIMO. All times reported include the total time from each method, including training, property calculation times, and generation times if applicable.

To run MolDQN for the property targeting task, which requires obtaining an optimized set of molecules, we used the last molecule of the most recent 1,000 training episodes to build a set on which success and diversity were calculated.

Experimental details. For the VAE, we use a 64-dimensional embedding layer that feeds into four batch-normalized 1,000-dimensional (2,000 for first layer) linear layers with ReLU activation. This generates a Gaussian output for the 1024-dimensional latent space that can be sampled from. For the decoder, we also use four batch-normalized linear layers with ReLU activation, with the same dimensions. Softmax is used over all possible symbols at each symbol location in the output layer, and the VAE is trained with evidence lower bound (ELBO), with the negative log-likelihood reconstruction term multiplied by 0.9 and the KL-divergence term multiplied by 0.1. The VAE is trained over 18 epochs with a learning rate of 0.0001 using the Adam optimizer.

For the property predictor, we use three 1,000-dimensional linear layers with ReLU activation. Layer width, number of layers, and activation function were determined after hyperparameter optimization for predicting penalized log P, and these hyperparameters were then used for all other property prediction tasks. Similarly, we did not tune baseline methods for specific tasks, and used only the default hyperparameters tuned on a single task. For each property to predict, we use PyTorch Lightning to choose the optimal learning rate with the Adam optimizer to train the predictor over 5 epochs, then perform backward optimization with a learning rate of 0.1 for 10 epochs.

All experiments, including baselines, were run on two GTX 1080 Ti GPUs, one for running PyTorch code and the other for running AutoDock-GPU, and 4 CPU cores with 32 GB memory.

Autodock-GPU. We use AutoDock-GPU, a GPU accelerated version of AutoDock4 with an additional AdaDelta local search method, to calculate binding affinities for LIMO. It is fast enough for our purposes while still generating reasonably accurate results (Santos-Martins et al., 2021).

To generate docking scores from a SMILES string produced by LIMO, we perform the following steps:

Generate grid files for docking using AutoGrid4. For human estrogen receptor, we set the bounding box for docking to include the well-known ligand binding site. For human peroxisomal acetyl-CoA acyl transferase 1, a novel target, we use fpocket (Guilloux et al., 2009) to predict the binding pocket and set the docking bounding box around it.

For each SMILES to evaluate, we convert it to a 3D .pdbqt file using obabel 2.4.0 (O'Boyle et al., 2011). We set pH=7.4 to assign hydrogens and set Gasteiger partial charges.

We run AutoDock-GPU (Santos-Martins et al., 2021) with default parameters on the .pdbqt files, in batch mode if applicable.

With the generated .dig files from AutoDock-GPU, we extract the top binding energy number in the results table.

Absolute-binding free energy. To corroborate our AutoDock-GPU predicted binding affinities, we conducted absolute binding free energy (ABFE) calculations on our most promising ligands. ABFE calculations estimate the binding free energy ΔG_{bind}, i.e., the difference between the free energy of a molecule's bound and unbound states, by computing the reversible work of moving a molecule from water into the binding site of the targeted protein. The dissociation constant is then obtained as K_{D}(ABFE)=e^{-ΔG}^{_{bind}}^{/RT}, where R is the gas constant and T is absolute temperature (Gilson & Zhou, 2007). The free energy calculation is done with detailed molecular dynamics simulations of the protein and the molecule dissolved in thousands of water molecules. This method is more detailed and computationally expensive, and typically more accurate, than docking (Cournia et al, 2020). Here, the 5 best-scoring poses from AutoDock-GPU were sent to the software BAT.py (Heinzelmann & Gilson, 2021) to compute the binding free energy, ΔGᵢ, for each pose i. The overall binding free energy accounting for all 5 poses was then obtained as ΔG_{bind}=−RT In i^{e-ΔG}^{ⱼ}^{/RT} (Gilson & Zhou, 2007). Note that the pose with the most favorable (negative) ΔGᵢ contributes the most to the overall binding free energy, and this is also the most stable and hence most probable binding pose of the ligand. We thus analyzed the protein-ligand interactions for this most stable pose. For each ligand, we use the mean free energy of two independent ABFE runs from calculations initiated with different random seeds.

Additional Experiments

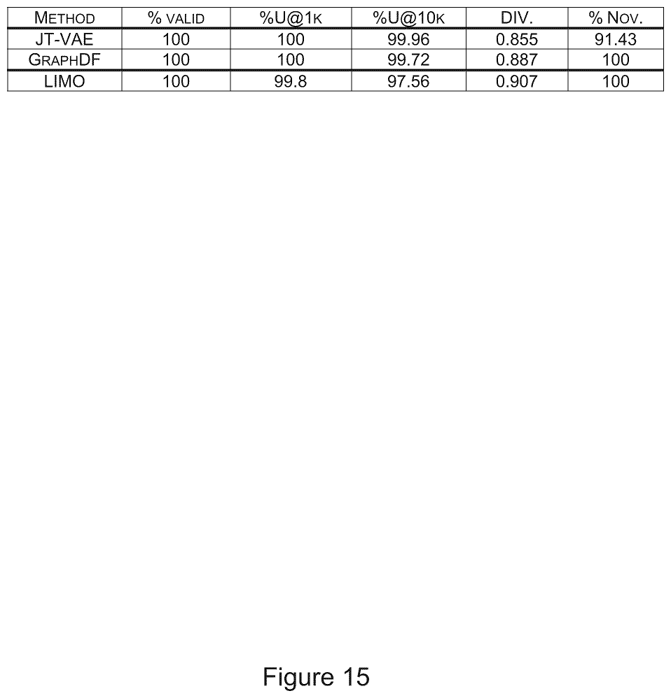

Random generation of molecules. Results from the random generation of 30,000 molecules are summarized in the table shown in FIG. 15. LIMO achieves the highest diversity score among compared methods, an important metric when using the latent space as a basis for the optimization of molecules on a wide range of properties. This diversity provides the foundation for LIMO's ability to generate a diverse set of molecules with desirable properties.

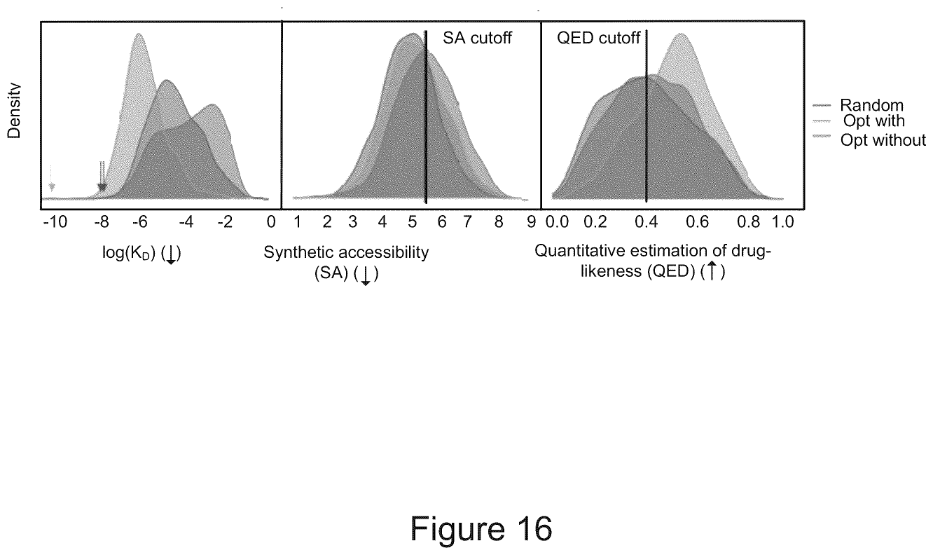

Justification of multi-objective optimization. FIG. 16 shows distributions of properties from randomly sampled molecules, molecules optimized on all three objectives (computed binding affinity against ESR1, QED, and SA), and optimized molecules leaving out one objective. We also show our QED and SA cutoff values used in the filtering step defined in Section 3.4. As shown, inclusion in the objective function pushes each property in the direction of improvement, or, in the case of SA, prevents it from decreasing more than it would have if it had not been included. Therefore, multi-objective optimization is successful in generating more molecules with potentially high binding affinity within the defined cutoff ranges, so is advantageous over single-objective optimization.

What is claimed is:

1. A method comprising:
obtaining, by one or more computing devices including one or more processors and memory, first molecules representation data that indicates, for individual first molecules, an arrangement of atoms;
generating, by at least a portion of the one or more computing devices and based on the first molecules representation data, second molecules representation data using one or more first neural networks, the second molecules representation data including a plurality of nodes with a first group of the plurality of nodes individually corresponding to a vector indicating a compressed version of a respective arrangement of atoms of an individual first molecule;
determining, by at least a portion of the one or more computing devices and based on a second molecules representation, a third molecule representation using one or more second neural networks, the third molecule representation indicating an additional arrangement of atoms of a third molecule;
determining, by at least a portion of the one or more computing devices and based on the third molecule representation, one or more values for one or more molecular properties of the third molecule using one or more additional computational models, at least one molecular property of the one or more molecular properties indicating a binding affinity to a target molecule; and
determining, by at least a portion of the one or more computing devices and based on the one or more values of the one or more molecular properties, a second group of the plurality of nodes included in the second molecules representation data using a gradient optimization technique, the second group of the plurality of nodes individually corresponding to a vector indicating an additional arrangement of atoms of an additional molecule having at least a threshold value of the at least one molecular property.

2. The method of claim 1, comprising:
obtaining, by at least a portion of the one or more computing devices, masking data indicating one or more positions of the third molecule that correspond to a substructure that is to remain fixed during a molecule optimization process;
generating, by at least a portion of the one or more computing devices and as part of the molecule optimization process, a plurality of modified molecule representations with individual modified molecule representations indicating a modified molecule that includes the substructure and includes one or more atoms outside of the substructure being modified from a respective third molecule of the third molecule; and
determining, by at least a portion of the one or more computing devices and using the one or more additional computational models, that an individual modified molecule that corresponds to a modified molecule representation of the plurality of modified molecule representations has an additional value for a molecular property of the one or more molecular properties that is greater than an initial value for the molecular property with respect to at least the third molecule that includes the substructure.

3. The method of claim 2, wherein the molecule optimization process with respect to the third molecule representation includes:
modifying, by at least a portion of the one or more computing devices, one or more atoms outside of the substructure of the third molecule representation to generate a respective modified molecule representation that corresponds to a respective modified molecule having an arrangement of atoms different from the additional arrangement of atoms;
determining, by at least a portion of the one or more computing devices and using the one or more additional computational models, a modified value for the molecular property of the modified molecule; and
determining, by at least a portion of the one or more computing devices, that the modified value for the molecular property of the modified molecule is greater than a value for the molecular property of the one or more molecular properties of the third molecule.

4. The method of claim 1, wherein the one or more additional computational models include:
a first computational model to generate one or more values of log P for the third molecule;
a second computational model to generate one or more values of a dissociation constant that corresponds to the target molecule with respect to the third molecule;
a third computational model to generate one or more values indicating an amount of sp3 hybridized carbons of the third molecule;
a fourth computational model to generate one or more values indicating one or more measures of similarity for the third molecule with respect to existing treatments for one or more biological conditions;
a fifth computational model to generate one or more values indicating non-specific activity with respect to a plurality of biological target molecules; and
a sixth computational model to generate one or more values indicating a measure of molecular novelty for the third molecule.

5. The method of claim 1, wherein the one or more first neural networks correspond to an encoder of a variational autoencoder and the one or more second neural networks correspond to a decoder of the variational autoencoder.

6. The method of claim 5, comprising:
performing, by at least a portion of the one or more computing devices, a training process for the variational autoencoder to minimize a reconstruction loss of the variational autoencoder.

7. The method of claim 6, wherein the training process comprises:
obtaining, by at least a portion of the one or more computing devices, a training data set indicating training molecules representation data that indicates, for individual training molecules, an arrangement of atoms using a string of characters;
generating, by at least a portion of the one or more computing devices, first training molecule representations using the encoder, individual first training molecule representations corresponding to a vector indicating a compressed version of the first training molecule representations;
generating, by at least a portion of the one or more computing devices and based on the first training molecule representations, one or more second training molecule representations using the decoder, individual second training molecule representations indicating a modified arrangement of atoms using a modified character string;
analyzing, by at least a portion of the one or more computing devices, the one or more second training molecule representations with respect to the training data set to determine a reconstruction loss for the variational autoencoder; and
modifying, by at least a portion of the one or more computing devices and based on the reconstruction loss, one or more computational layers of at least one of the encoder or the decoder to minimize a loss function of the variational autoencoder.

8. The method of claim 7, wherein the loss function of the variational autoencoder is minimized using negative log likelihood.

9. The method of claim 6, comprising:
generating, by at least a portion of the one or more computing devices, a plurality of further molecule representations using the decoder and based on a plurality of vectors extracted from the plurality of nodes; and
performing, by at least a portion of the one or more computing devices, an additional training process of the one or more additional computational models using the plurality of further molecule representations to predict values of the one or more molecular properties of molecules that correspond to the plurality of further molecule representations.

10. The method of claim 9, wherein:
the one or more additional computational models are trained after the variational autoencoder is trained; and
computational layers of the variational autoencoder remain unchanged during the additional training process of the one or more additional computational models.

11. The method of claim 9, wherein the one or more additional computational models are trained to predict binding affinity to one or more regions of the target molecule.

12. A method comprising:
obtaining, by one or more computing devices including one or more processors and memory, first molecules representation data that indicates, for individual first molecules, an arrangement of atoms using a string of characters;
generating, by at least a portion of the one or more computing devices and based on the first molecules representation data, second molecules representation data using one or more first neural networks, the second molecules representation data including a plurality of nodes with at least a portion of individual nodes of the plurality of nodes corresponding to a vector indicating a compressed version of a respective arrangement of atoms of an individual first molecule;
determining, by at least a portion of the one or more computing devices and based on one or more vectors included in the second molecules representation data, third molecules representation data using one or more second neural networks, the third molecules representation data indicating, for individual third molecules, an additional arrangement of atoms using an additional string of characters;
determining, by at least a portion of the one or more computing devices and based on the third molecules representation data, one or more values for one or more molecular properties of one or more third molecules using one or more additional computational models, at least one molecular property of the one or more molecular properties indicating a binding affinity to a target molecule;
obtaining, by at least a portion of the one or more computing devices, masking data indicating one or more positions of the one or more third molecules that correspond to a substructure that is to remain fixed during a molecule optimization process;
generating, by at least a portion of the one or more computing devices and as part of the molecule optimization process, a plurality of additional molecule representations with individual additional molecule representations indicating an additional molecule that includes the substructure and includes one or more atoms outside of the substructure being modified from a respective third molecule of the one or more third molecules; and
determining, by at least a portion of the one or more computing devices and using the one or more additional computational models, that an individual molecule that corresponds to an additional molecule representation of the plurality of additional molecule representations has an additional value for a molecular property of the one or more molecular properties that is greater than an initial value for the molecular property with respect to at least a portion of the one or more third molecules that include the substructure.

13. The method of claim 12, wherein the one or more additional computational models are trained to predict binding affinity to one or more regions of the target molecule.

14. The method of claim 12, wherein the plurality of additional molecule representations correspond to molecules having molecular weights no greater than 800 Daltons.

15. A system comprising:
one or more hardware processors; and
memory storing computer-readable instructions that, when executed by the one or more hardware processors, cause the one or more hardware processors to perform operations comprising:
obtaining first molecules representation data that indicates, for individual first molecules, an arrangement of atoms using a string of characters;
generating, based on the first molecules representation data, second molecules representation data using one or more first neural networks, the second molecules representation data including a plurality of nodes with at least a portion of individual nodes of the plurality of nodes corresponding to a vector indicating a compressed version of a respective arrangement of atoms of an individual first molecule;
determining, based on one or more vectors included in the second molecules representation data, third molecules representation data using one or more second neural networks, the third molecules representation data indicating, for individual third molecules, an additional arrangement of atoms using an additional string of characters;
determining, based on the third molecules representation data, one or more values for one or more molecular properties of one or more third molecules using one or more additional computational models, at least one molecular property of the one or more molecular properties indicating a binding affinity to a target molecule;
obtaining masking data indicating one or more positions of the one or more third molecules that correspond to a substructure that is to remain fixed during a molecule optimization process;
generating, as part of the molecule optimization process, a plurality of additional molecule representations with individual additional molecule representations indicating an additional molecule that includes the substructure and includes one or more atoms outside of the substructure being modified from a respective third molecule of the one or more third molecules; and
determining, using the one or more additional computational models, that an individual molecule that corresponds to an additional molecule representation of the plurality of additional molecule representations has an additional value for a molecular property of the one or more molecular properties that is greater than an initial value for the molecular property with respect to at least a portion of the one or more third molecules that include the substructure.

16. The system of claim 15, wherein the memory stores additional computer-readable instructions that, when executed by the one or more hardware processors, cause the one or more hardware processors to perform additional operations comprising:
determining, using an additional computational model of the one or more additional computational models and based on one or more additional molecule representations of the plurality of additional molecule representations, first values for a first metric for one or more additional molecules that correspond to the one or more additional molecule representations, the first values of the first metric indicating measures of similarity between the one or more additional molecules and one or more further molecules that are provided as treatments for one or more biological conditions; and
determining, using an additional computational model of the one or more additional computational models and based on the one or more additional molecule representations of the plurality of additional molecule representations, second values for a second metric for the one or more additional molecules, the second values for the second metric indicating a likelihood of synthesizing the one or more additional molecules under one or more sets of conditions.

17. The system of claim 16, wherein the memory stores additional computer-readable instructions that, when executed by the one or more hardware processors, cause the one or more hardware processors to perform additional operations comprising:
determining a subset of the one or more additional molecules having at least a threshold value for the first metric and at least an additional threshold value for the second metric.

18. The system of claim 15, wherein plurality of nodes are included in a latent space that corresponds to the second molecules representation data.

19. The system of claim 18, wherein:
a subset of the plurality of additional molecule representations correspond to a subset of the plurality of nodes located in the latent space, and the subset of the plurality of additional molecule representations correspond to a first group of additional molecules having values for the one or more molecular properties; and
the memory stores additional computer-readable instructions that, when executed by the one or more hardware processors, cause the one or more hardware processors to perform additional operations comprising:
moving, according to a gradient-based computational technique, to a different subset of the plurality of nodes of the latent space outside of the subset of the plurality of nodes;
determining an additional group of second molecules representations that corresponds to the different subset of the plurality of nodes;
determining, using the one or more second neural networks and based on the additional group of second molecules representations, additional third molecules representations that indicate additional third molecules; and
determining, using the one or more additional computational models and based on the additional third molecules, further values for the molecular property.

20. The system of claim 15, wherein the memory stores additional computer-readable instructions that, when executed by the one or more hardware processors, cause the one or more hardware processors to perform additional operations comprising:
modifying one or more atoms of an additional molecule corresponding to an additional molecular representation to generate a modified molecular representation that corresponds to a modified molecule having an arrangement of atoms different from the additional molecule;
determining, using the one or more additional computational models, a modified value for the molecular property of the modified molecule; and
determining that modified value for the molecular property of the modified molecule is greater than the additional value for the molecular property of the additional molecule.
